(19) United States
(12) Patent Application Publication
(10) Pub. No.: US 2009/0299155 A1
(43) Pub. Date: Dec. 3, 2009

(54) CONTINUOUS CARDIAC MARKER SENSOR SYSTEM

(75) Inventors: Richard C. Yang, Irvine, CA (US); Bradley Shigeto Matsubara, Escondido, CA (US); Robert Boock, San Diego, CA (US)

Correspondence Address: KNOBBE, MARTENS, OLSEN & BEAR, LLP, 2040 MAIN STREET, FOURTEENTH FLOOR, IRVINE, CA, 92614, US

(73) Assignee: DexCom, Inc., San Diego, CA (US)

(21) Appl. No.: 12/362,194

(22) Filed: Jan. 29, 2009

Related U.S. Application Data
(60) Provisional application No. 61/024,841, filed on Jan. 30, 2008.

(51) Int. Cl. A61B 5/0205; A61B 5/145

(52) U.S. Cl. 600/301; 600/508; 600/309

(57) ABSTRACT

The present invention relates generally to systems and methods for continuous measurement of a cardiac marker in vivo. In some embodiments, the system includes a continuous sensor and a communication device. The continuous sensor is configured to continuously measure a concentration of a cardiac marker in vivo and to provide a signal associated therewith. The communication device includes a processor module configured to process the signal to obtain cardiac information, wherein the communication device is configured to output the cardiac information.

### CROSS-REFERENCE TO RELATED APPLICATIONS

This application claims the benefit under 35 U.S.C. §119(e) of U.S. provisional application Ser. No. 61/024,841, filed Jan. 30, 2008, the disclosure of which is hereby expressly incorporated by reference in its entirety and is hereby expressly made a portion of this application.

### FIELD OF THE INVENTION

The preferred embodiments relate generally to continuous detection and/or measurement of cardiac markers in vivo.

### BACKGROUND OF THE INVENTION

Diseases of the heart are a major cause of death & disability worldwide. As of 2007, heart disease is the leading cause of death in the United States, England, and Wales, killing one person every 34 seconds in the United States alone. A variety of acute and chronic cardiac problems (e.g., ischemic cardiac injury, infarction, angina, inflammation of the heart and surrounding tissue due to infection, autoimmune disease, etc., arrhythmia, post-surgical problems, K⁺ and/or Ca²⁺ imbalances, and the like) require an increasing number of emergency room visits and hospital admissions. Given that heart diseases primarily affect an older population, the costs of medical care, for those afflicted with heart disease, are likely to increase.

In addition to testing and/or monitoring with various cardiac diagnostic devices, serum cardiac marker concentrations are generally measured one or more times during the patient's visit, as part of the process of diagnosing, evaluating and/or monitoring the cardiac patient's condition. Obtaining the cardiac marker concentrations is currently a slow process that can take several hours, requiring blood collection and analysis in a clinical laboratory. Accordingly, cardiac marker concentrations are measured only intermittently, about 1-3 times per day during the patient's stay.

### SUMMARY OF THE INVENTION

In a first aspect, a system for continuously detecting a cardiac marker is provided, the system comprising: a continuous sensor configured to continuously, continually, and/or intermittently measure a concentration of a cardiac marker in vivo and provide a signal associated therewith; and a communication device comprising a processor module configured to process the signal to obtain cardiac information, wherein the communication device is configured to output the cardiac information.

In an embodiment of the first aspect, the cardiac marker is selected from the group consisting of creatine kinase MB, cardiac troponin T, cardiac troponin I, troponin C, aspartate transaminase, lactate dehydrogenase, myoglobin, alanine transaminase, alkaline phosphatase, albumin, ischemia-modified albumin, myeloperoxidase, glycogen phosphorylase isoenzyme BB, brain natriuretic peptide, N-terminal pro-natriuretic peptide, monocyte chemo attractive protein, gamma glutamyl transpeptidase, high sensitive C-reactive protein, heart type fatty acid binding protein, P-selectin, soluble CD40 ligand, glycoprotein IIb/IIIa, prothrombin fragment 1.2, D-dimer, thrombin-antithrombin II, beta-thromboglobulin, platelet factor 4, platelet/endothelial cell adhesion molecule 1, soluble fibrin, glycogen phosphorylase-BB, thrombus precursor protein, interleukin-1 receptor family/ST2, interleukin 6, interleukin 18, placental growth factor, pregnancy-associated plasma protein A, glutathione peroxidase, plasma thioredoxin, Cystatin C, serum deoxyribonuclease I, ATP/ADP, total bilirubin, direct bilirubin, potassium, calcium, and combinations thereof.

In an embodiment of the first aspect, the cardiac information is selected from the group consisting of a cardiac marker concentration, a change in cardiac marker concentration, an acceleration of cardiac marker concentration change, an area under the curve of a plot of time versus cardiac marker concentration, and combinations thereof.

In an embodiment of the first aspect, the communication device is configured to provide one or more alarms indicative of cardiac health.

In an embodiment of the first aspect, the processor module is configured to trigger the alarm when the cardiac marker concentration meets a criterion.

In an embodiment of the first aspect, the processor module is configured to provide a cardiac status.

In an embodiment of the first aspect, the cardiac status comprises a level of cardiac status.

In an embodiment of the first aspect, the processor module is configured to predict a cardiac status.

In an embodiment of the first aspect, the cardiac status is selected from the group consisting of improving cardiac health, declining cardiac health, stable cardiac health, ischemic heart disease, pericarditis, endocarditis, myocarditis, congestive cardiac failure, cardiogenic shock, acute coronary syndrome, alcoholic cardiomyopathy, coronary artery disease, congenital heart disease, ischemic cardiomyopathy, hypertensive cardiomyopathy, valvular cardiomyopathy, inflammatory cardiomyopathy, cardiomyopathy secondary to a systemic metabolic disease, dilated cardiomyopathy, hypertrophic cardiomyopathy, arrhythmogenic right ventricular cardiomyopathy, restrictive cardiomyopathy, noncompaction cardiomyopathy, congestive heart failure, valvular heart disease, hypertensive heart disease, and combinations thereof.

In an embodiment of the first aspect, the processor module is configured to predict a cardiac event.

In an embodiment of the first aspect, the cardiac event is selected from the group consisting of myocardial infarction, myocardial ischemic attack, unstable angina, acute coronary syndrome, myocardial rupture, endocarditis, pericarditis, cardiogenic shock, and combinations thereof.

In an embodiment of the first aspect, the system further comprises a vascular access device configured for insertion into at least one of a circulatory system of the host and an extracorporeal blood circulation device.

In an embodiment of the first aspect, the sensor is further configured to continuously, continually, and/or intermittently measure a second substance in vivo and to provide a signal associated therewith.

In an embodiment of the first aspect, the second substance is selected from the group consisting of glucose, potassium, calcium, oxygen, carbon dioxide, and liver enzymes.

In an embodiment of the first aspect, the communication device is configured to receive and process data from a secondary medical device, such as an electrocardiograph, an oxygen monitor, a fluid delivery device, a pacing device, leads, a mechanical ventilator, an extracorporeal membrane oxygenator, a cardiac output monitor, a blood pressure monitor, a central venous pressure monitor, a pulmonary capillary wedge pressure monitor, an intra-aortic balloon pump, an end-tidal carbon dioxide monitor, an intra-cranial pressure monitor, a Doppler monitor, a thermometer, a hemodynamic monitor, a patient monitor, and combinations thereof.

In an embodiment of the first aspect, the communication device is configured to display data from the secondary medical device.

In an embodiment of the first aspect, the communication device is configured to transmit instructions to a secondary medical device.

In an embodiment of the first aspect, the secondary medical device displays the cardiac information.

In an embodiment of the first aspect, the communication device comprises a user interface configured to display the cardiac information.

In an embodiment of the first aspect, the user interface is remote.

In an embodiment of the first aspect, the user interface is configured to provide an alarm.

In an embodiment of the first aspect, the communication device comprises a component of a secondary medical device, such as an electrocardiograph, an oxygen monitor, a fluid delivery device, a pacing device, leads, a mechanical ventilator, an extracorporeal membrane oxygenator, a cardiac output monitor, a blood pressure monitor, a central venous pressure monitor, a pulmonary capillary wedge pressure monitor, an intra-aortic balloon pump, an end-tidal carbon dioxide monitor, an intra-cranial pressure monitor, a Doppler monitor, a thermometer, a hemodynamic monitor, a patient monitor, and combinations thereof.

In an embodiment of the first aspect, the system is configured to calibrate the signal using at least one reference data point.

In an embodiment of the first aspect, the system is configured to calibrate the signal using at least one reference point for each of two or more cardiac markers.

In a second aspect, a method for determining cardiac health of a host is provided, the method comprising: using a sensor to continuously, continually, and/or intermittently detect a concentration of a cardiac marker in vivo; providing a signal associated with the concentration of the cardiac marker; processing the signal to obtain cardiac information; and outputting the cardiac information.

In an embodiment of the second aspect, the cardiac marker is selected from the group consisting of creatine kinase MB, cardiac troponin T, cardiac troponin I, troponin C, aspartate transaminase, lactate dehydrogenase, myoglobin, alanine transaminase, alkaline phosphatase, albumin, ischemia-modified albumin, myeloperoxidase, glycogen phosphorylase isoenzyme BB, brain natriuretic peptide, N-terminal pro-natriuretic peptide, monocyte chemo attractive protein, gamma glutamyl transpeptidase, high sensitive C-reactive protein, heart type fatty acid binding protein, P-selectin, soluble CD40 ligand, glycoprotein IIb/IIIa, prothrombin fragment 1.2, D-dimer, thrombin-antithrombin II, beta-thromboglobulin, platelet factor 4, platelet/endothelial cell adhesion molecule 1, soluble fibrin, glycogen phosphorylase-BB, thrombus precursor protein, interleukin-1 receptor family/ST2, interleukin 6, interleukin 18, placental growth factor, pregnancy-associated plasma protein A, glutathione peroxidase, plasma thioredoxin, Cystatin C, serum deoxyribonuclease I, ATP/ADP, total bilirubin, direct bilirubin, potassium, calcium, and combinations thereof.

In an embodiment of the second aspect, the processing step comprises providing a cardiac status.

In an embodiment of the second aspect, the cardiac status comprises a level of cardiac status.

In an embodiment of the second aspect, the processing step comprises predicting a future cardiac status.

In an embodiment of the second aspect, the processing step comprises predicting a cardiac event.

In an embodiment of the second aspect, the outputting step comprises displaying the cardiac information.

In an embodiment of the second aspect, the outputting step comprises providing one or more alarms.

In an embodiment of the second aspect, the method further comprises the step of calibrating the signal.

### BRIEF DESCRIPTION OF THE DRAWINGS

FIG. 1a is a block diagram illustrating a continuous cardiac marker sensor system 10, in one embodiment.

FIG. 1b is a graph illustrating CK-MB and troponin concentration changes over time, after injury to the cardiac muscle.

FIG. 2 is a block diagram illustrating components of a communication device 110, in one embodiment.

FIG. 3 is a flow chart illustrating a method of using a continuous cardiac marker sensor system 10, in one embodiment.

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

### DEFINITIONS

In order to facilitate an understanding of the preferred embodiments, a number of terms are defined below.

The term “A/D Converter” as used herein is a broad term, and is to be given its ordinary and customary meaning to a person of ordinary skill in the art (and it is not to be limited to a special or customized meaning), and refers without limitation to hardware and/or software that converts analog electrical signals into corresponding digital signals.

The term “alarm,” as used herein is a broad term and is to be given its ordinary and customary meaning to a person of ordinary skill in the art (and is not to be limited to a special or customized meaning), and furthermore refers without limitation to a signal or indication related to an occurrence of an event and/or condition related to the host.

The term “analyte” as used herein is a broad term, and is to be given its ordinary and customary meaning to a person of ordinary skill in the art (and it is not to be limited to a special or customized meaning), and refers without limitation to a substance or chemical constituent in a biological fluid (for example, blood, interstitial fluid, cerebral spinal fluid, lymph fluid or urine) that can be analyzed. Analytes may include naturally occurring substances, artificial substances, metabolites, and/or reaction products. In some embodiments, the analyte for measurement by the sensor heads, devices, and methods disclosed herein is a cardiac marker. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotimidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobinopathies A, S, C, and E, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17 alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids may also constitute analytes in certain embodiments. The analyte may be naturally present in the biological fluid, for example, a metabolic product, a hormone, an antigen, an antibody, and the like. Alternatively, the analyte may be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body may also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and glucose.

The term “area under the curve,” as used herein is a broad term and is to be given its ordinary and customary meaning to a person of ordinary skill in the art (and is not to be limited to a special or customized meaning), and furthermore refers without limitation to the area under the curve of a graph of Y versus X. For example, in some embodiments, the size of the area under the curve of a graph of concentration of a cardiac marker versus time is indicative of the magnitude of a cardiac condition and/or event. In a further embodiment, the area under the curve of a graph of CK-MB concentration versus time is indicative of the extent (e.g., magnitude/level) of a myocardial infarction (MI).

The term “cardiac marker,” also referred to as “myocardial markers,” as used herein is a broad term and is to be given its ordinary and customary meaning to a person of ordinary skill in the art (and is not to be limited to a special or customized meaning), and furthermore refers without limitation to a substance that is released into and/or elevated in the circulatory system (e.g., blood, serum, plasma, etc.) in conjunction with an upcoming, present, or recent cardiac disease, insult, injury, and the like (e.g., ischemia, myocardial infarction, pericarditis, cardiac infection, ischemic and/or coagulative necrosis, acute coronary syndrome, etc.) and/or as a result thereof. In some embodiments, cardiac markers include, but are not limited to cardiac troponin T (cTnT), cardiac troponin I (cTnI), troponin C (TnC), creatine kinase MB (CK-MB), aspartate transaminase (AST), lactate dehydrogenase (LDH), myoglobin (MB or MYO), alanine transaminase (ALT), aspartate transaminase (AST), alkaline phosphatase (ALP), albumin (Alb), gamma glutamyl transpeptidase (GGT), high sensitive C-reactive protein (hsCRP), heart type fatty acid binding protein (H-FABP), myeloperoxidase (MPO), brain natriuretic peptide (BNP), P-selectin (soluble and membrane bound), soluble CD40 ligand (sCD40L), glycoprotein IIb/IIIa (GPIIb/IIIa), prothrombin fragment 1.2 (PTF1.2), D-dimer (DD), thrombin-antithrombin II (TAT), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), interleukin-1 receptor family/ST2, interleukin 6 (IL-6), interleukin 12 (IL-12), interleukin 18 (IL-18), placental growth factor (P1GF), pregnancy-associated plasma protein A (PAPP-A), glutathione peroxidase, plasma thioredoxin, Cystatin C, serum deoxyribonuclease I, and ATP/ADP, human Fas ligand (hFasL), total bilirubin (TBIL) and direct bilirubin, potassium (K⁺) and calcium (Ca²⁺), and blood gases (O₂, CO₂). In preferred embodiments, a cardiac marker is an analyte measured by a continuous sensor configured to measure the concentration of the cardiac marker in vitro and/or in vivo.

The term “cardiac event,” as used herein is a broad term and is to be given its ordinary and customary meaning to a person of ordinary skill in the art (and is not to be limited to a special or customized meaning), and furthermore refers without limitation to an occurrence that involves and/or affects the heart at a given time, such as but not limited to a myocardial infarction or an ischemic event.

The term “insult,” as used herein is a broad term and is to be given its ordinary and customary meaning to a person of ordinary skill in the art (and is not to be limited to a special or customized meaning), and furthermore refers without limitation to an injury, attack or trauma.

The terms “cardiac status” and “cardiac health,” as used herein are a broad terms and are to be given their ordinary and customary meaning to a person of ordinary skill in the art (and are not to be limited to a special or customized meaning), and furthermore refer without limitation to the state and/or condition of the heart, an extent of cardiac well-being. In some circumstances, cardiac status and/or health can be improving or declining, improved or worse, or unchanged relative to the host's state/health at a previous time. In some circumstances, a host's cardiac status/health can be compared to that of an average person of a similar age, sex, group, and the like. In some circumstances, cardiac status and/or cardiac health can be categorized into levels of severity, such that patients can be segregated accordingly. A level can be associated with certain therapeutic procedures. “Future cardiac status/health” refers to a predicted cardiac status/health.

The term “catheter” as used herein is a broad term, and is to be given its ordinary and customary meaning to a person of ordinary skill in the art (and are not to be limited to a special or customized meaning), and refers without limitation to a tube that can be inserted into a host's body (e.g., cavity, duct or vessel). In some circumstances, catheters allow drainage or injection of fluids or access by medical instruments or devices. In some embodiments, a catheter is a thin, flexible tube (e.g., a “soft” catheter). In alternative embodiments, the catheter can be a larger, solid tube (e.g., a “hard” catheter). In some embodiments, a catheter can have a single lumen or multiple lumens. The term “cannula” is interchangeable with the term “catheter” herein.

The term “circulatory system of the host,” as used herein is a broad term and is to be given its ordinary and customary meaning to a person of ordinary skill in the art (and is not to be limited to a special or customized meaning), and furthermore refers without limitation to the organs and tissues involved in circulating blood and lymph through the body.

The terms “continuous” and “continuously” as used herein are broad terms, and are to be given their ordinary and customary meanings to a person of ordinary skill in the art (and are not to be limited to a special or customized meaning), and refer without limitation to the condition of being marked by substantially uninterrupted extension in space, time or sequence. In one embodiment, an analyte concentration is measured continuously, continually, and/or intermittently (regularly or irregularly) for example at time intervals ranging from fractions of a second up to, for example, 1, 2, 5, or 10 minutes, or longer. For example, continuous cardiac marker measurement systems generally continually measure cardiac marker concentration without required user initiation and/or interaction for each measurement. These terms include situations wherein data gaps can exist (e.g., when a continuous sensor is temporarily not providing data, or when data from the continuous sensor is disregarded or not considered).

The phrase “continuous analyte sensing” as used herein is a broad term, and is to be given its ordinary and customary meaning to a person of ordinary skill in the art (and it is not to be limited to a special or customized meaning), and refers without limitation to the period in which monitoring of analyte concentration (e.g., cardiac marker concentration) is continuously, continually, and/or intermittently (regularly or irregularly) performed, for example, at time intervals ranging from fractions of a second up to, for example, 1, 2, 5, or 10 minutes, or longer.

The term “counts” as used herein is a broad term, and is to be given its ordinary and customary meaning to a person of ordinary skill in the art (and it is not to be limited to a special or customized meaning), and refers without limitation to a unit of measurement of a digital signal. In one example, a raw data stream measured in counts is directly related to a voltage (for example, converted by an A/D converter), which is directly related to current from a working electrode.

The term “communication device,” as used herein is a broad term and is to be given its ordinary and customary meaning to a person of ordinary skill in the art (and is not to be limited to a special or customized meaning), and furthermore refers without limitation to a device configured to communicate information. In some embodiments, the output is to a display (bedside or remote therefrom).

The term “criterion,” as used herein is a broad term and is to be given its ordinary and customary meaning to a person of ordinary skill in the art (and is not to be limited to a special or customized meaning), and furthermore refers without limitation to a basis for comparison; a reference point against which other things can be evaluated. In some embodiments, a criterion is associated with an action, instruction, command, and the like, that the system performs and/or provides when a criterion has been (or has not been) met. As a non-limiting example, the system can be configured such that when the concentration of a cardiac marker increases by 200% an alarm is sounded. In other embodiments, the criterion has two or more conditions that must be met before the associated action is taken. In some embodiments, the system is configured to compare data to two or more criteria, wherein each criterion is associated with a task to be performed. In some embodiments, a plurality of “criteria” must be met, wherein each of the criteria includes one or more conditions. For example, if conditions A and B have been satisfied, then alarm #1 is sounded, while, if condition C is met, then a text message is sent to a remote monitoring station. In some embodiments, a criterion has a single condition that must be met.

The terms “computer” or “computer system” as used herein are broad terms, and are to be given their ordinary and customary meanings to a person of ordinary skill in the art (and are not to be limited to a special or customized meaning), and refer without limitation to a machine that can be programmed to manipulate data.

The term “electronics” as used herein is a broad term, and is to be given its ordinary and customary meaning to a person of ordinary skill in the art (and is not to be limited to a special or customized meaning), and refers without limitation to electronic circuitry configured to measure, process, receive, and/or transmit data.

The term “extracorporeal (blood) circulation device,” as used herein is a broad term and is to be given its ordinary and customary meaning to a person of ordinary skill in the art (and is not to be limited to a special or customized meaning), and furthermore refers without limitation to a device configured to circulate at least a portion of the host's blood outside of his body. In one exemplary embodiment, an extracorporeal (blood) circulation device includes a shunt, such as an arterial-vascular shunt (AV-shunt). Additional exemplary embodiments, of an extracorporeal (blood) circulation device can include, but are not limited to, a dialysis machine, a cardiopulmonary bypass pump (a.k.a., heart-lung machine), and bedside blood chemistry/gas analysis devices.

The term “fluid delivery device,” as used herein is a broad term and is to be given its ordinary and customary meaning to a person of ordinary skill in the art (and is not to be limited to a special or customized meaning), and furthermore refers without limitation to a device configured to deliver a fluid to the host, such as a pump (e.g., a pump system) configured to deliver IV fluid and/or medicament(s) to a host via a catheter.

The term “host” as used herein is a broad term, and is to be given its ordinary and customary meaning to a person of ordinary skill in the art (and it is not to be limited to a special or customized meaning), and refers without limitation to plants or animals, for example humans.

The term “level of cardiac status/health,” as used herein is a broad term and is to be given its ordinary and customary meaning to a person of ordinary skill in the art (and is not to be limited to a special or customized meaning), and furthermore refers without limitation to quantification (and/or categorization) of a host's cardiac status/health, wherein each level is associated with one or more characteristics and/or criteria.

The term “medical device” as used herein is a broad term, and is to be given its ordinary and customary meaning to a person of ordinary skill in the art (and are not to be limited to a special or customized meaning), and refers without limitation to an instrument, apparatus, implement, machine, contrivance, implant, in vitro reagent, or other similar or related article, including a component part or accessory which is intended for use in the diagnosis of disease or other conditions, or in the cure, mitigation, treatment, or prevention of disease, in man or other animals, or intended to affect the structure or any function of the body of man or other animals.

The terms “operably connected” and “operably linked” as used herein are broad terms, and are to be given their ordinary and customary meaning to a person of ordinary skill in the art (and they are not to be limited to a special or customized meaning), and refer without limitation to one or more components being linked to another component(s) in a manner that allows transmission of signals between the components. These terms are broad enough to include wired and wireless connectivity.

The term “output,” as used herein is a broad term and is to be given its ordinary and customary meaning to a person of ordinary skill in the art (and is not to be limited to a special or customized meaning), and furthermore refers without limitation to presentation of host data by the present system, such as but not limited to the host, a caretaker, a component of the system or a secondary medical device integrated with the system. Output can include, but is not limited to, raw data, processed data, cardiac information, instructions to the host, a caretaker or a secondary medical device, and the like. In some circumstances, data and/or information received from (or input by) the host, a caretaker, and/or a secondary medical device can be output by the system.

The term “potentiostat” as used herein is a broad term, and is to be given its ordinary and customary meaning to a person of ordinary skill in the art (and it is not to be limited to a special or customized meaning), and refers without limitation to an electrical system that applies a potential between the working and reference electrodes of a two- or three-electrode cell at a preset value and measures the current flow through the working electrode. A potentiostat can include multiple channels, such that potentials can be applied to two or more working electrode-reference electrode pairs. Typically, the potentiostat forces whatever current is necessary to flow between the working and reference or counter electrodes to keep the desired potential, as long as the needed cell voltage and current do not exceed the compliance limits of the potentiostat.

The terms “processor module” and “processor” as used herein are broad terms, and are to be given their ordinary and customary meaning to a person of ordinary skill in the art (and are not to be limited to a special or customized meaning), and refer without limitation to a computer system, state machine, processor, and the like designed to perform arithmetic or logic operations using logic circuitry that responds to and processes the basic instructions that drive a computer.

The term “RAM” as used herein is a broad term, and is to be given its ordinary and customary meaning to a person of ordinary skill in the art (and it is not to be limited to a special or customized meaning), and refers without limitation to a data storage device for which the order of access to different locations does not affect the speed of access. RAM is broad enough to include SRAM, for example, which is static random access memory that retains data bits in its memory as long as power is being supplied.

The terms “raw data stream” and “data stream” signal as used herein are broad terms, and are to be given their ordinary and customary meaning to a person of ordinary skill in the art (and they are not to be limited to a special or customized meaning), and refer without limitation to an analog or digital signal directly related to the analyte concentration measured by the analyte sensor. In one example, the raw data stream is digital data in “counts” converted by an A/D converter from an analog signal (for example, voltage or amps) representative of an analyte concentration. The terms broadly encompass a plurality of time spaced data points from a substantially continuous analyte sensor, which comprises individual measurements taken at time intervals ranging from fractions of a second up to, for example, 1, 2, or 5 minutes or longer. In some embodiments, raw data includes one or more values (e.g. digital value) representative of the current flow integrated over time (e.g. integrated value), for example, using a charge counting device, or the like.

The term “RF transceiver” as used herein is a broad term, and is to be given its ordinary and customary meaning to a person of ordinary skill in the art (and it is not to be limited to a special or customized meaning), and refers without limitation to a radio frequency transmitter and/or receiver for transmitting and/or receiving signals.

The term “ROM” as used herein is a broad term, and is to be given its ordinary and customary meaning to a person of ordinary skill in the art (and it is not to be limited to a special or customized meaning), and refers without limitation to read-only memory, which is a type of data storage device manufactured with fixed contents.

The term “secondary medical device,” as used herein is a broad term and is to be given its ordinary and customary meaning to a person of ordinary skill in the art (and is not to be limited to a special or customized meaning), and furthermore refers without limitation to another or auxiliary medical device distinct from a primary medical device. For example, in some embodiments, a continuous cardiac marker sensor system is integrated with a secondary medical device, such as but not limited to an ECG, a pressure transducer, a cardiac pacing device, a ventilator, a pump for delivering fluids and/or medicaments to the host, a hemodynamic monitor, a patient monitor, and the like.

The terms “substantial” and “substantially” as used herein are broad terms, and are to be given their ordinary and customary meaning to a person of ordinary skill in the art (and are not to be limited to a special or customized meaning), and refer without limitation to a sufficient amount that provides a desired function. For example, an amount greater than 50 percent, an amount greater than 60 percent, an amount greater than 70 percent, an amount greater than 80 percent, or an amount greater than 90 percent.

The term “vascular access device” as used herein is a broad term, and is to be given its ordinary and customary meaning to a person of ordinary skill in the art (and are not to be limited to a special or customized meaning), and refers without limitation to any device that provides access (e.g., operable communication, fluid communication) to the host's vascular system. Some vascular access devices, such as a syringe needle, generally provide short-term (e.g., minutes or hours) access to the host's vascular system. Other vascular access devices, such as a Swan-Ganz pulmonary catheter, generally provide access to the host's vascular system for a longer period of time (e.g., hours, days, weeks, or longer). Some vascular access devices, such as an A-V shunt for dialysis, can be implanted substantially permanently in the host's vascular system. While vascular access devices are generally manufactured from materials separate from the host's body, some vascular access devices, such as a fistula, can be formed from a portion of the host's vascular system itself Vascular access devices include but are not limited to catheters, cannula, shunts, blood withdrawal devices, connectors and/or valves for connecting a catheter to tubing (e.g., a Leur lock, T-connector, Y-connector, etc.) and the like.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

### Overview

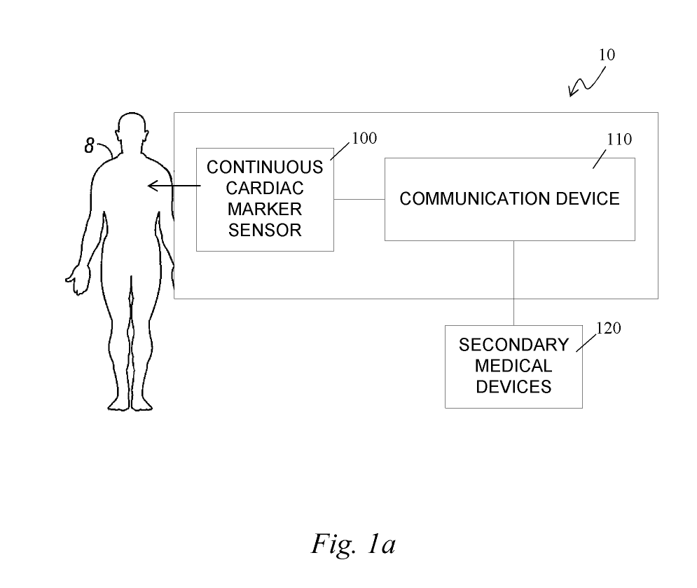

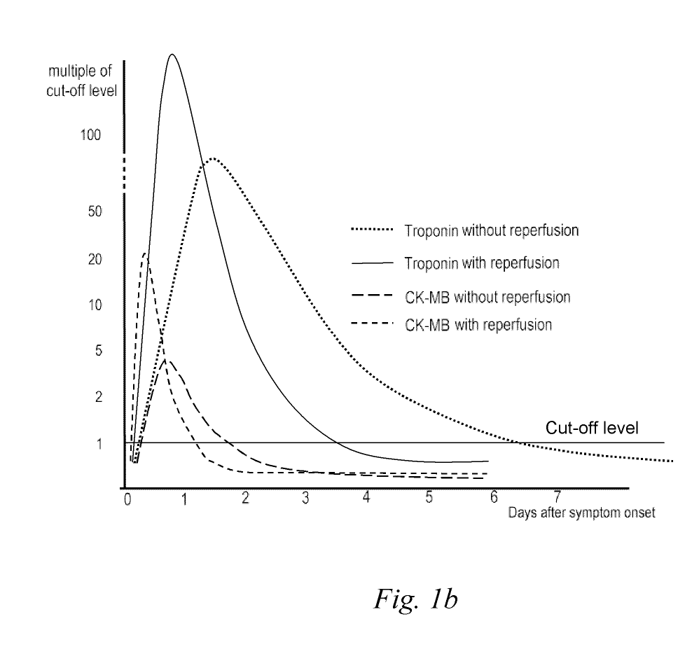

Referring to FIG. 1a, the preferred embodiments provide a system 10 for continuously detecting a cardiac marker in vivo, including at least one continuous analyte sensor 100 configured to continuously measure a concentration of a cardiac marker and to provide a signal associated therewith, and a communication device 110. The communication device 110 includes a processor module configured to process the signal to obtain cardiac information, and to output the cardiac information. In some embodiments, the continuous cardiac marker sensor 100 is configured for insertion into the host's 8 circulatory system, such as via a vascular access device, such as but not limited to, an arterial catheter having one or more lumens. In preferred embodiments, the sensor 100 is operably connected to the communication device 110, which can include a bedside or remotely located receiver (e.g., including a display and/or an alarm). In some embodiments, the communication device is configured to process the signal from the sensor in order to provide real-time monitoring of the host's condition, to provide an alarm when host data passes a threshold and/or meets a criterion, to aid in diagnosis of the host's cardiac health, and to predict impending cardiac events, such as but not limited to a myocardial infarction (MI), which thereby enables preventive and/or palliative measures to be taken to prevent and/or lessen the cardiac event. In preferred embodiments, the continuous cardiac marker sensor system 10 is configured for functional integration (e.g., operable connection) with one or more secondary medical devices 120, such as but not limited to an ECG, an intra-arterial pressure monitor, a balloon pump, a fluid delivery device, a bedside blood chemistry device, a ventilator, a patient monitor, and the like. Components of the sensor system are discussed in greater detail elsewhere herein.

### System Components

Continuous Analyte Sensor

FIG. 1a is a block diagram illustrating one embodiment of the continuous cardiac marker sensor system 10. In general, the preferred embodiments provide a continuous analyte sensor 100 that measures a concentration of an analyte of interest or a substance indicative of the concentration or presence of the analyte, such as a cardiac marker. In some embodiments, the analyte sensor is an invasive, minimally invasive, or non-invasive device, for example a subcutaneous, transdermal, intravascular, or extracorporeal device. In some embodiments, the analyte sensor can be configured to analyze a plurality of intermittent biological samples. The analyte sensor can be configured to use any method of analyte-measurement known in the art, including enzymatic, chemical, physical, electrochemical, spectrophotometric, polarimetric, calorimetric, radiometric, and the like.

As a non-limiting example, in some embodiments, the analyte sensor 100 is a continuous electrochemical analyte sensor configured to provide at least one working electrode and at least one reference electrode, which are configured to measure a signal associated with a concentration of the analyte in the host, such as described in more detail below. The output signal is typically a raw data stream that is used to provide a useful value of the measured analyte concentration in a host to the patient or doctor, for example. However, the analyte sensors of some embodiments comprise at least one additional working electrode configured to measure at least one additional signal, as discussed elsewhere herein. For example, in some embodiments, the additional signal is associated with the baseline and/or sensitivity of the analyte sensor, thereby enabling monitoring of baseline and/or sensitivity changes that may occur over time. In some embodiments, the analyte sensor is configured to measure two or more analytes, such as but not limited to two or more cardiac markers, or a cardiac marker and glucose.

In general, electrochemical continuous analyte sensors define a relationship between sensor-generated measurements (for example, current in pA, nA, or digital counts after A/D conversion) and a reference measurement (for example, glucose concentration mg/dL or mmol/L) that are meaningful to a user (for example, patient or doctor). For example, in the case of an implantable diffusion-based glucose oxidase electrochemical glucose sensor, the sensing mechanism generally depends on phenomena that are linear with glucose concentration, for example: (1) diffusion of glucose through a membrane system (for example, biointerface membrane and membrane system) situated between implantation site and/or the electrode surface, (2) an enzymatic reaction within the membrane system, and (3) diffusion of the H₂O₂ to the sensor. Because of this linearity, calibration of the sensor can be understood by solving an equation:

y=mx+b

wherein y represents the sensor signal (e.g. counts), x represents the estimated glucose concentration (e.g. mg/dL), m represents the sensor sensitivity to glucose (e.g. counts/mg/dL), and b represents the baseline signal (e.g. counts). When both sensitivity m and baseline (background) b change over time in vivo calibration has generally requires at least two independent, matched data pairs (x₁, y₁; x₂, y₂) to solve for m and b and thus allow glucose estimation when only the sensor signal, y is available. Matched data pairs can be created by matching reference data (for example, one or more reference glucose data points from a blood glucose meter, or the like) with substantially time corresponding sensor data (for example, one or more glucose sensor data points) to provide one or more matched data pairs, such as described in co-pending U.S. Patent Publication No. US-2005-0027463-A1. In some implantable glucose sensors, such as described in more detail in U.S. Pat. No. 6,329,161 to Heller et al., which is incorporated herein by reference in its entirety, the sensing layer utilizes immobilized mediators (e.g. redox compounds) to electrically connect the enzyme to the working electrode, rather than using a diffusional mediator. In some implantable glucose sensors, such as described in more detail in U.S. Pat. No. 4,703,756, the system has two oxygen sensors situated in an oxygen-permeable housing, one sensor being unaltered and the other contacting glucose oxidase allowing for differential measurement of oxygen content in bodily fluids or tissues indicative of glucose levels. A variety of systems and methods of measuring glucose in a host are known, all of which may benefit from some of all of the preferred embodiments to provide a sensor having a signal-to-noise ratio that is not substantially affected by non-constant noise.

In preferred embodiments, the continuous analyte sensor 100 is configured to continuously measure a concentration of a cardiac marker in vivo, and to provide a signal associated therewith. In general, cardiac markers are substances that can be found in the circulatory system, wherein their concentration in the blood can be correlated with the host's cardiac health. Cardiac markers are discussed in greater detail elsewhere herein. In some embodiments, the sensor is configured to detect more than one analyte. In one exemplary embodiment, the sensor is configured to continuously detect at least two cardiac markers. In another exemplary embodiment, the sensor is configured to continuously detect a cardiac marker and glucose. This embodiment enables both monitoring of the host's cardiac health and tight control of glucose levels, which is known to be critical to patient outcome in a critical care medical setting, especially for diabetic hosts.

In some embodiments, the sensor is configured to continuously measure a second substance (e.g., a second analyte, in addition to the cardiac marker) in vivo and to provide a signal associated therewith. For example, the second substance can be a second cardiac marker, glucose, potassium, calcium, oxygen, carbon dioxide, liver enzymes, and the like. An extensive list of possible analytes is provided in the “Definitions” section. While not wishing to be bound by theory, it is believed that monitoring a second substance (e.g., a secondary analyte) can provide additional information useful in determining (in real-time) the host's cardiac status, the host's cardiac health, predicting future cardiac events, providing therapy, and the like. As a non-limiting example, Level 1 can be associated with characteristics A, B and C, while Level 2 is associated with characteristics D, E, and F. Thus, according to this example, a host exhibiting characteristics A, B and C can be classified as a “Level 1” patient. In some embodiments, a host's cardiac “level” can be used as a diagnostic gauge, such as for determining therapy or a prognosis for the host. For example, the NYHA and American College of Cardiology/American Heart Association staging systems are frequently used to triage chest pain patients.

By way of example and not of limitation, a wide variety of suitable detection methods, such as but not limited to enzymatic, chemical, physical, electrochemical, immunochemical, optical, radiometric, calorimetric, protein binding, and microscale methods of detection, can be employed in the preferred embodiments, although any other techniques can be used in alternate embodiments. Additional description of analyte sensor configurations and detection methods can be found in U.S. Patent Publication No. US-2007-0213611-A1, U.S. Patent Publication No. US-2007-0027385-A1, U.S. Patent Publication No. US-2005-0143635-A1, U.S. Patent Publication No. US-2007-0020641-A1, U.S. Patent Publication No. US-2007-0020641-A1, and U.S. Patent Publication No. US-2005-0196820-A1, U.S. Pat. No. 5,517,313, U.S. Pat. No. 5,512,246, U.S. Pat. No. 6,400,974, U.S. Pat. No. 6,711,423, U.S. Pat. No. 7,308,292, U.S. Pat. No. 7,303,875, U.S. Pat. No. 7,289,836, U.S. Pat. No. 7,289,204, U.S. Pat. No. 5,156,972, U.S. Pat. No. 6,528,318, U.S. Pat. No. 5,738,992, U.S. Pat. No. 5,631,170, U.S. Pat. No. 5,114,859, U.S. Pat. No. 7,273,633, U.S. Pat. No. 7,247,443 U.S. Pat. No. 6,007,775, U.S. Pat. No. 7,074,610, U.S. Pat. No. 6,846,654, U.S. Pat. No. 7,288,368, U.S. Pat. No. 7,291,496, U.S. Pat. No. 5,466,348, U.S. Pat. No. 7,062,385 U.S. Pat. No. 7,244,582, U.S. Pat. No. 7,211,439, U.S. Pat. No. 7,214,190, U.S. Pat. No. 7,171,312, U.S. Pat. No. 7,135,342, U.S. Pat. No. 7,041,209, U.S. Pat. No. 7,061,593, U.S. Pat. No. 6,854,317, U.S. Pat. No. 7,315,752, and U.S. Pat. No. 7,312,040, each of which is incorporated herein by reference, in its entirety.

In general, the continuous cardiac marker sensors of the preferred embodiments are configured for non-ambulatory use, such as when the host is confined to a bed or chair in the hospital or clinic. However, the system, or at least some of its components, can be configured for ambulatory use. In some embodiments, the system is configured such that a portion of the non-ambulatory continuous cardiac marker sensor system can be disconnected (e.g., from the rest of the system) and moved with the host from one location to another. In one exemplary embodiment, wherein the host is in the ICU and needs to be moved to the operating room (OR), and wherein the sensor is inserted into an artery via a vascular access device, the sensor is configured to be disconnected from the communication device in the hospital room and subsequently connected to a another communication device at another location, such as in the OR, thus preventing sensor removal and insertion of a new sensor at a later time. Connection of the system components is discussed in greater detail elsewhere herein. In some embodiments, the sensor system (or at least a component thereof) is configured for ambulatory use.

Cardiac Markers

In general, cardiac markers are substances that can be measured in the host's blood and are indicative of the occurrence of a cardiac event (e.g., a myocardial infarction, MI), cardiac status and/or cardiac health. In some embodiments, cardiac markers are proteins from cardiac tissue found in the blood. Cardiac markers are sometimes referred to as “cardiac enzymes.” While many cardiac markers are enzymes (e.g., CK-MB, troponins, AST, etc.), some cardiac markers are not enzymes (e.g., IL-6, ATP/ADP, K⁺, etc.). Cardiac markers are released into the bloodstream when damage to the heart (or other components of the circulatory system) occurs, as in the case of a myocardial infarction. For example, when damage to the heart occurs, levels of cardiac markers generally rise over time. In some circumstances, a cardiac marker concentration falls outside (either above or below) a normal concentration range in conjunction with a cardiac event in a host. Accordingly, changes in the concentrations of one or more cardiac markers can be correlated with the cardiac status and/or cardiac health of the patient.

FIG. 1b illustrates the changes in the concentrations of two exemplary cardiac markers (CK-MB and troponin), after damage to the heart, such as by a myocardial infarction. Within a few days of the damage, the concentrations of CK-MB and troponin rise and then gradually taper off over the next several days. CK-MB peaks within about 6-24 hours and then rapidly declines, while troponin peaks within about 1-2 days and is measurable for about 3-7 days. Concentrations of CK-MB and troponin drop more rapidly with reperfusion (reestablishment of blood flow to a damaged tissue or organ), as compared to concentration changes without reperfusion, indicating increased healing with reperfusion. While not wishing to be bound by theory, it is believed that changes in the concentrations of one or more cardiac markers, measured by continuous sensing (e.g., by a continuous cardiac marker sensor system), correlate with cardiac status and cardiac health, and can be predictive of cardiac events.

In preferred embodiments, the system is configured to detect at least one cardiac marker, including but are not limited to troponin (cTnT, cTnI, TnC), creatine kinase MB (CK-MB), aspartate transaminase (AST), lactate dehydrogenase (LDH), myoglobin (MB), alanine transaminase (ALT), aspartate transaminase (AST), alkaline phosphatase (ALP), albumin (Alb), gamma glutamyl transpeptidase (GGT), high sensitive C-reactive protein (hsCRP), heart type fatty acid binding protein (H-FABP), myeloperoxidase (MPO), brain natriuretic peptide (BNP), P-selectin (soluble and membrane bound), soluble CD40 ligand (sCD40L), glycoprotein IIb/IIIa (GPIIb/IIIa), prothrombin fragment 1.2 (PTF1.2), D-dimer (DD), thrombin-antithrombin II (TAT), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), interleukin-1 receptor family/ST2, interleukin 6 (IL-6), interleukin 18 (IL-18), placental growth factor (PIGF), pregnancy-associated plasma protein A (PAPP-A), glutathione peroxidase, plasma thioredoxin, cystatin C, serum deoxyribonuclease I, and ATP/ADP, total bilirubin (TBIL) and direct bilirubin. In some embodiments, cardiac markers include but are not limited to non-protein substances, such as serum potassium (K⁺) and calcium (Ca²⁺), some carbohydrates, lipids and nucleic acids, and the like. Additional markers of cardiac status/health include but are not limited to blood gases (O₂, CO₂), liver enzymes (or their reaction products), and glucose.

Vascular Access Device

In general, the continuous cardiac marker sensor 100 is configured for insertion into the host's circulatory system and/or into an extracorporeal medical device (e.g., an extracorporeal blood circulation device) via a vascular access device, such as but not limited to a sterile catheter and/or cannula. In some embodiments, the catheter (e.g., single-lumen or multi-lumen) is configured as a component of an insertion set, which may include sterile tubing, a support (e.g., a needle) configured to support the catheter during insertion into the host's vascular system, and optionally one or more tubing connectors and/or valves. In some embodiments, the vascular access device is configured for arterial insertion and includes one or more lumens. In some embodiments, the catheter is a pulmonary artery catheter (a catheter configured for insertion into a pulmonary artery, e.g., a Swan-Ganz catheter). Pulmonary artery catheters allow direct, simultaneous measurement of pressures in the right atrium, right ventricle, pulmonary artery, and the filling pressure (“wedge” pressure) of the left atrium, and are generally used in a critical care setting to detect heart failure or sepsis, monitor therapy, and evaluate the effects of drugs. A standard Swan-Ganz catheter has two lumens, but catheters with more lumens (e.g., 5, 6 or more) are compatible with the sensor system 10. Medical devices that can be used in conjunction with various embodiments of the analyte sensor system include any monitoring device requiring placement in a human vessel, duct or body cavity, a dialysis machine, a heart-lung bypass machine, blood collection equipment, a blood pressure monitor, an automated blood chemistry analysis device and the like.

In some embodiments, the vascular access device is a venous catheter configured for insertion into a vein or an extracorporeal device, such as but not limited to an A-V shunt, a pressure monitor, a dialysis machine, and/or a fluid delivery system. In these embodiments, the catheter includes one or more lumens. Advantageously, multi-lumen catheters permit concurrent access to the host's circulatory system by two or more devices, and reduce the number of insertion procedures the host must endure.

In some embodiments, at least a portion of the sensor is configured for insertion through the vascular access device after the vascular access device has been inserted into the host's circulatory system. In a further embodiment, the sensor is configured for insertion through the vascular access device and into the host's circulatory system proper, such that at least a portion of the sensor resides within the host's artery/vein/heart. In another further embodiment, the sensor is configured to be disposed within the lumen of the vascular access device and blood samples are drawn up into the lumen, such that the sensor sufficiently contacts the blood sample and analyte measurements can be performed therein. In some embodiments, the system is configured to detect two or more analytes (e.g., cardiac markers). For example, in some embodiments, the system is configured with a multilumen vascular access device (e.g., a multilumen catheter) and two or more continuous cardiac marker sensors. In some embodiments, the sensor is configured to detect at least two cardiac markers and is inserted into one lumen of the multilumen catheter, for example. In a further embodiment, the sensor is configured to detect glucose. In some embodiments, the system is configured for use with at least one single-analyte cardiac marker sensor and at least one multi-analyte cardiac marker sensor. In one exemplary embodiment, the system is configured for use with a continuous CK-MB sensor and a second sensor configured to detect both cardiac troponin T and aspartate transaminase. In another exemplary embodiment, the sensor is configured to detect at least three cardiac markers, such as but not limited to CK-MB, troponins, myoglobin, and/or brain natriuretic peptide. In yet another exemplary embodiment, the sensor is configured to detect at least three cardiac markers and at least one additional substance, such as O₂, K⁺, liver enzymes, and the like. In some embodiments, the sensor is an integral portion of the vascular access device, such as on a surface of the vascular access device (e.g., exterior, interior, tip). In a further embodiment, a plurality of sensors and/or sensor electrodes can be located on at least one surface of the vascular access device. In some embodiments, one or more sensors and/or sensor electrodes are located within the lumen of a connector, such as but not limited to a Leur-lock connector, which is configured to connect an inserted catheter to tubing. In this embodiment, the system is configured to pull back the sample into the Leur-lock, such that the sensor(s) is contacted with the sample. One skilled in the art appreciates the wide variety of configurations that can be used with the instant system.

In some embodiments, the system is configured such that blood samples are withdrawn from the host (e.g., at regular intervals) and contacted with the sensor extracorporeally. For example, the sensor can be disposed within a connector (e.g., T-, X- or Y-connector) attached to a vascular access device via tubing, such that a blood sample is drawn all the way back through the tubing and into the connector, where the sample contacts a sufficient portion of the sensor for an analyte measurements to be made. In some embodiments, the system is configured to return the sample to the host, such that there is substantially no loss of blood volume to the host. In some embodiments, the blood is disposed of as waste and is therefore not returned to the host. In other embodiments, the system is configured to withdraw a sample from the host, to separate an aliquot from the sample (for testing), and then return the unused portion of the sample to the host. In a further embodiment, the aliquot is tested and then disposed of as waste. In some circumstances, these procedures can involve an undesirable blood volume loss. However, collection of smaller samples (e.g., 100 μl versus 1 ml) and/or less frequent samples (e.g., once per hour versus every 30-minutes) can minimize blood volume loss.

In some embodiments, the system is configured to dialyze the withdrawn samples, such that the dialysate is tested for the presence of one or more cardiac markers. In some embodiments, the system is configured to test the dialysate within about 1-5 minutes post collection. In some other embodiments, the system is configured to pool the dialysate over time, such as but not limited to over a period of 5, 10, 15, 20, 25 or 30-minutes, or longer. When the fluid collection period is complete, the system tests the pooled dialysate for the presence and/or concentration of one or more cardiac markers. In one exemplary embodiment, the system is configured to microdialyze the sample. Microdialysis employs a semi-permeable membrane to exclude molecules based on size (e.g., molecular weight); only those molecules that are sufficiently small can pass through the membrane. In another exemplary embodiment, a microdialysis membrane is applied to the continuous analyte sensor. Accordingly, sufficiently small molecules (e.g., determined by the microdialysis membrane's molecular weight cut off) pass through the membrane to be tested by the analyte sensor. Advantageously, in addition to the removal cellular material, dialysis can be configured to remove a wide variety of undesired blood components, thereby boosting the cardiac marker signal during testing and reducing false positives. Accordingly, dialysis can provide increased accuracy in cardiac marker detection (e.g., when compared to whole blood testing).

In some embodiments, the system 10 is operably connected to and/or integrated with a secondary medical device 120, such as another medical device useful to monitor and/or treat the host's illness. Some secondary medical device may require access to the host's circulatory system (e.g., a fluid delivery device, an arterial blood pressure monitor, a bedside blood gas monitor, an arterial balloon pump), while other secondary medical devices will not require such access (e.g., an ECG or a ventilator). In some embodiments, the system can be integrated with medical devices used in the operating room, such as a cardiopulmonary bypass machine or anesthesia equipment.

In some embodiments, the secondary medical device is operably connected to the sensor system. In some embodiments, at least a portion of the secondary medical device can perform one or more functions of the sensor system (e.g., data processing, analysis, output, etc.). In some embodiments, a secondary medical device can provide data to the present system, and/or receive data and/or instructions from the present system. In some embodiments, the sensor system can perform one or more functions of the secondary medical device, such as but not limited to data processing and output. In some embodiments, a component of the sensor system (e.g., the communications device) can include a component of a secondary medical device. Examples of secondary medical devices include but are not limited to a pressure transducer, a pump for delivering IV fluids and/or medicaments to the host, a bedside blood chemistry monitor, an ECG, an oxygen monitor, a carbon dioxide monitor, a pace maker, leads, an intra-aortic balloon pump, a mechanical ventilator, a Doppler cardiac monitor, a hemodynamic monitor, a patient monitor, and a display. A secondary medical device can be functionally attached and/or integrated with the present system by wired and/or wireless means.

Additional descriptions of insertion of sensors into vascular access devices can be found in U.S. Patent Publication No. US-2008-0119703-A1, U.S. Patent Publication No. US-2008-0119704-A1, U.S. Patent Publication No. US-2008-0119706-A1, U.S. Patent Publication No. US-2008-0108942-A1, U.S. Patent Publication No. US-2008-0086042-A1, U.S. Patent Publication No. US-2008-0086044-A1, and U.S. Patent Publication No. US-2008-0086273-A1, each of which is incorporated herein by reference in its entirety.

Communication Device

Referring again to FIG. 1a, in preferred embodiments, the sensor system 10 includes a communication device 110 that is operably connected to the continuous cardiac marker sensor 100 and optionally to a secondary medical device 120. In preferred embodiments, the system 10 includes electronics, also referred to as a “computer system” that can include hardware, firmware, and/or software that enable measurement and processing of data associated with analyte levels in the host. Portions of the electronics associated with the communication device are configured to receive and process sensor data and providing an output of cardiac information (including storing information), and can reside on the sensor, a housing located adjacent to the sensor, on a vascular access device (and tubing and/or components connected thereto), on a bedside device, and/or on a remote device located remotely from the host's physical location, such as at a nurse's station, a doctor's office, a clinical lab or a medical records department. In one exemplary embodiment, the electronics include a potentiostat (e.g., single and/or multi-channel), a power source for providing power to the sensor, and other components useful for signal processing. In another exemplary embodiment, the electronics include an RF module for transmitting data from sensor electronics to a receiver remote from the sensor. In another exemplary embodiment, the sensor electronics are wired to a receiver, which records the data and optionally transmits the data to a remote location, such as but not limited to a nurse's station, for tracking the host's progress and to alarm the staff if a therapy is required. In some embodiments, the output is to a secondary medical device. In some embodiments, the communication device is further configured to receive data and/or information from a secondary medical device and to optionally process the data and/or information. In some embodiments, the output includes instructions for a secondary medical device. In various embodiments, the communication device comprises at least a portion of sensor electronics and/or a processor module.

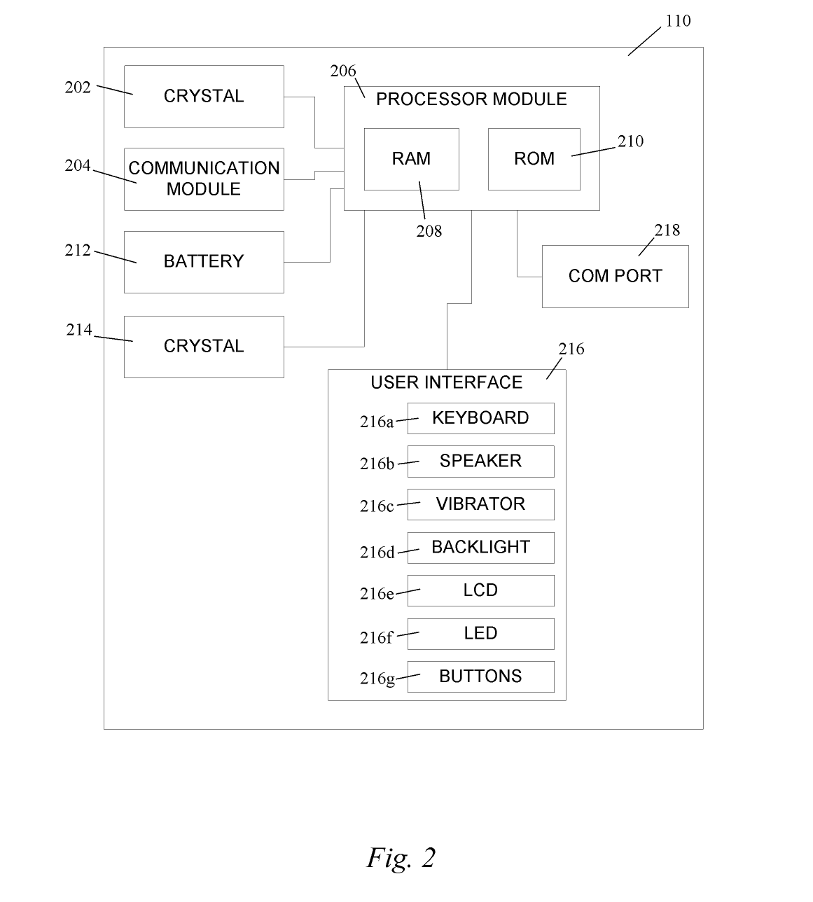

FIG. 2 is a block diagram that illustrates some of the electronics/components of the communication device 110 of the sensor system 10, which includes the electronics necessary for running the sensor 100, collecting and processing data, and outputting the cardiac information. Components of the communication device can be disposed on or proximal to the sensor, such as but not limited to disposed on the vascular access device, on a connector configured to couple the vascular access device to tubing, tubing to tubing, tubing to a fluid container, on a valve, and the like. In some embodiments, only a portion of the electronics (e.g., the potentiostat) is disposed on the sensor (e.g., proximal to the sensor), while the remaining electronics are disposed remotely from the sensor, such as on a stand or by the bedside. In a further embodiment, a portion of the electronics can be disposed in a central location, such as a nurse's station.

In additional embodiments, some or all of the electronics can be in wired or wireless communication with the sensor 100 and/or other portions of the communication device 110, or a secondary medical device 120. For example, a potentiostat disposed on the sensor and/or sensor housing can be wired to the remaining electronics (e.g., a processor module 206, a communication module 204, a recorder, a transceiver, etc.), which reside on the bedside. In another example, some portion of the electronics is wirelessly connected to another portion of the electronics, such as by infrared (IR) or RF. In one embodiment, a potentiostat resides on a tubing connector and/or valve and is connected to a receiver by RF; accordingly, a battery, RF transmitter, and/or other minimally necessary electronics are provided with the tubing connector and/or valve and the receiver includes an RF transceiver.

A battery 212 can be operably connected to the communication device 110 and provide the power for the sensor 100 or to another system component. In one embodiment, the battery is a lithium manganese dioxide battery; however, any appropriately sized and powered battery can be used (for example, AAA, nickel-cadmium, zinc-carbon, alkaline, lithium, nickel-metal hydride, lithium-ion, zinc-air, zinc-mercury oxide, silver-zinc, and/or hermetically-sealed). In some embodiments, the battery is rechargeable, and/or a plurality of batteries can be used to power the system. In some embodiments, a quartz crystal 214 is operably connected to the processor module 206 and maintains system time for the computer system as a whole, for example for the programmable acquisition time within the processor module. Alternatively, the system can be configured to plug into an electrical outlet.

A communication module 204 can be operably connected to the processor module 206 and transmit the sensor data from the sensor to a receiver via a wireless or wireless transmission. In some embodiments, mechanisms, such as RF telemetry, optical, infrared radiation (IR), ultrasonic, or the like, can be used to transmit and/or receive data.

Typically, the electronics include a processor module 206 that includes a central control unit that controls the processing of the sensor system 10. In some embodiments, the processor module includes a microprocessor, however a computer system other than a processor can be used to process data as described herein, for example an ASIC can be used for some or all of the sensor's central processing. For example, in some embodiments, the system is configured with an ASIC, wherein the ASIC includes at least RAM, programming memory and data storage memory (not shown). In some embodiments, the processor module typically provides semi-permanent storage of data, for example, storing data such as sensor identifier (ID) and programming to process data streams (for example, programming for data smoothing and/or replacement of signal artifacts such as is described in U.S. Patent Publication No. US-2005-0043598-A1). The processor module additionally can be used for the system's cache memory, for example for temporarily storing recent sensor data. In some embodiments, the processor module comprises memory storage components such as ROM 208, RAM 210, dynamic-RAM, static-RAM, non-static RAM, rewritable ROMs, non-volatile memory (e.g., EEPROM, flash memory, etc.), and the like.

In some embodiments, the processor module 206 comprises a digital filter, for example, an infinite impulse response (IIR) or finite impulse response (FIR) filter, configured to smooth the raw data stream from the A/D converter. Generally, digital filters are programmed to filter data sampled at a predetermined time interval (also referred to as a sample rate). In some embodiments, wherein the potentiostat is configured to continuously measure the analyte, for example, using a current-to-frequency converter, the processor module can be programmed to request a digital value from the A/D converter at a predetermined time interval, also referred to as the acquisition time. In these alternative embodiments, the values obtained by the processor are advantageously averaged over the acquisition time due the continuity of the current measurement.

In some embodiments, the processor further performs the processing, such as storing data, analyzing data streams, calibrating analyte sensor data, estimating analyte values, comparing estimated analyte values with time corresponding measured analyte values, analyzing a variation of estimated analyte values, downloading data, and controlling the user interface by providing analyte values, prompts, messages, warnings, alarms, and the like. In such cases, the processor includes hardware and software that performs the processing described herein, for example flash memory provides permanent or semi-permanent storage of data, storing data such as sensor ID, and programming to process data streams (for example, programming for performing estimation and other algorithms described elsewhere herein) and random access memory (RAM) stores the system's cache memory and is helpful in data processing. Alternatively, some portion of the data processing (such as described with reference to the processor elsewhere herein) can be accomplished at another (e.g., remote) processor and can be configured to be in wired or wireless connection therewith.

In preferred embodiments, the communication device 110 includes an output module, which is integral with and/or operatively connected with the processor 206, and includes programming for generating output based on the data stream received from the sensor system and it's processing incurred in the processor. In preferred embodiments, output is generated via a user interface 216 configured to display the cardiac information. In some embodiments, a user interface 216 is provided integral with (e.g., on the patient inserted medical device), proximal to (e.g., a receiver near the medical device including bedside or on a stand), or remote from (e.g., at a central station such as a nurse's station) the sensor electronics, wherein the user interface includes a keyboard 216a, a speaker 216b, a vibrator 216c, a backlight 216d, an LCD 216e or one or more LEDs 216f, and/or one or more buttons 216g. For example, in some embodiments, some of the user interface components can be proximal to the sensor, while other components of the user interface can be located remotely from the host. For example, a user interface including a display and buttons can be located on sensor housing or at the bedside while a second display and a speaker are located at the nurse's station. The components that comprise the user interface include controls to allow interaction of the user (e.g., the medical personnel) with the sensor system 10. The keyboard can allow, for example, input of user information, such as mealtime, exercise, medicament administration, customized therapy recommendations, and reference analyte values. The speaker can produce, for example, audible signals or alerts for conditions such as present and/or estimated ischemic or irregular pacing conditions (e.g., recurrent myocardial infarction, stent or conduit occlusion, atrial fibrillation, ventricular tachycardia, etc.). The vibrator can provide, for example, tactile signals or alerts for reasons such as described with reference to the speaker, above. The backlight can be provided, for example, to aid a user in reading the LCD in low light conditions. The LCD can be provided, for example, to provide the user with visual data output. In some embodiments, the LCD is a touch-activated screen, enabling each selection by a user, for example, from a menu on the screen. The buttons can provide for toggle, menu selection, option selection, mode selection, and reset, for example. In some alternative embodiments, a microphone can be provided to allow for voice-activated control.

In some embodiments, prompts or messages are displayed on the user interface 216 to convey information to the user (e.g., the medical personnel), such as current cardiac marker concentration, graph of cardiac marker concentration over time, current and/or predicted cardiac status and/or level, therapy recommendations, deviation of the measured analyte values from the estimated analyte values, alarms, and the like. Additionally, prompts can be displayed to guide the user through calibration, trouble-shooting of the calibration, integration with a secondary medical device 120, or delivery of a therapy.

Additionally, data output from the communications device can provide wired or wireless, one- or two-way communication between the user interface and a secondary medical device 120 (sometimes referred to as an external device). In some embodiments, the system 10 is configured to display cardiac information on a secondary medical device (e.g., on the user interface of the secondary medical device). In some embodiments, the system 10 is configured to display secondary medical device data/information (e.g., data/information from the secondary medical device) on the system's user interface 216. The secondary medical device can be any device that wherein interfaces or communicates with the sensor system 10, such as via wired or wireless communication. In some embodiments, the secondary medical device is a computer, and the system 10 is able to download historical data for retrospective analysis by a nurse or physician, for example. In some embodiments, the secondary medical device is a modem or other telecommunications station, and the system is able to send alerts, warnings, emergency messages, and the like, via telecommunication lines to a party remote from the host, such as a doctor or family member. In some embodiments, the secondary medical device is a medicament and/or fluid delivery pump, and the system 10 is able to communicate therapy recommendations, such as medicament amount and time to the pump. The secondary medical device can include other technology or medical devices, for example pacemakers, implanted analyte sensor patches, other infusion devices, telemetry devices, and the like. In some embodiments, the communications device includes a component of a secondary medical device.

The user interface 216, including keyboard, buttons, a microphone (not shown), and optionally the external device, can be configured to allow input of data. Data input can be helpful in obtaining information about the host (for example, meal time, medicament administration, respiration, function of the heart and the like), receiving instructions from a physician (for example, customized therapy recommendations, targets, criteria, thresholds, and the like), receiving calibration information, and downloading software updates, for example. Keyboard, buttons, touch-screen, and microphone are all examples of mechanisms by which a user (e.g., medical personnel) can input data directly into the system. A server, personal computer, personal digital assistant, medicament pump, and insulin pen are examples of external devices that can provide useful information to the receiver. Other devices internal or external to the sensor that measure other aspects of a patient's body (for example, temperature sensor, accelerometer, heart rate monitor, oxygen monitor, and the like) can be used to provide input helpful in data processing. In one embodiment, the user interface 216 can prompt the medical personnel to select an activity most closely related to the host's present activity, such as medication taken, surgical procedures, and the like, which can be helpful in linking to an individual's physiological patterns, or other data processing. In another embodiment, a temperature sensor and/or heart rate monitor can provide information helpful in linking activity, metabolism, and cardiac status/health of a host. While a few examples of data input have been provided here, a variety of information can be input, which can be helpful in data processing.

In some embodiments, the communication device is configured to provide one or more alarms indicative of the host's cardiac health (e.g., status and/or well-being). For example, a first alarm can indicate that the host's health has improved to a certain level; which can trigger a change in the host's management, such as but not limited to changes in medicament delivery, weaning from a respirator or removal of an intra-aortic balloon pump. In another example, a second alarm can indicate an impending cardiac event, such as such as a myocardial infarction or an ischemic attack, and to alarm the medical personnel to that intervention can be provided to prevent or lessen the extent of the impending event.

In some embodiments, an alarm is visual (e.g., illumination and/or blinking of a light, transmission of a message to a display such as a screen), auditory (e.g., a buzzer or bell, transmission to an auditory device such as a telephone), vibratory (a portion of the system shakes, such as is used with pagers and cellular telephones), or combinations thereof. In some embodiments, a plurality of alarms can be used, wherein each alarm is related to a different host condition and/or event. For example, a first alarm can be associated with a first condition, and a second alarm can be associated with a second condition. In some embodiments, an alarm is associated with a particular event, such as but not limited to predication of a pending myocardial infarction.

In a further embodiment, the processor module is configured to trigger an alarm when the cardiac marker concentration (or other cardiac information) meets a criterion. For example, the processor module can be configured to compare the cardiac marker concentration to a threshold, wherein if the concentration passes the threshold, an alarm is given, such as but not limited to displaying a text message, providing an auditory alarm (e.g., ringing, buzzing, etc.), flashing/blinking lights, and the like, at the bedside and/or remotely, and combinations thereof. In some embodiments, the alarm includes instructions to the caretaker, to perform a given task or take certain actions, such as but not limited to increasing/decreasing a medicament, performing an additional test, providing calibration information, or consulting with a supervising/lead physician, and the like. In some embodiments, the system is configured to provide instructions for a therapy to the caretaker, a secondary medical device, and the like. In a further embodiment, the therapy can be delivered manually or automatically, depending upon the system configuration. In some embodiments, a criterion is associated with an action, instruction, command, and the like, that the system is configured to perform and/or provide when a criterion has been (or has not been) met. As a non-limiting example, the system can be configured such that when the concentration of a cardiac marker increases by 200% and alarm is sounded. For example, if conditions A and B have been satisfied, then alarm #1 is sounded, while, if condition C is met, then a text message is sent to a remote monitoring station. In some embodiments, a criterion has a single condition that must be met. In other embodiments, the criterion has two or more conditions that must be met before the associated action is taken. In some embodiments, a plurality of “criteria” must be met, wherein each of the criteria includes one or more conditions. As a non-limiting example, in some embodiments, the system is configured to continuously measure the concentrations of at least two cardiac markers, and includes a criterion that the concentration of each cardiac markers must increase by at least one predetermined amount (e.g., a percentage) relative to the concentrations at a previous time period (e.g., at the time the host was admitted, within the past 24-hours, etc.), before an alarm is provided to the caretaker and/or at the host's bedside.

In some embodiments, the processor module is configured to provide a cardiac status, such as but not limited to a level of cardiac status. Alternatively or additionally, the processor module is configured to predict a cardiac status, in some embodiments. In one exemplary embodiment, the processor intelligently tracks (e.g., monitors) the cardiac information, and changes therein, and relates that information to one or more criteria, wherein, when the criteria are met, a cardiac status can be predicted. For example, the processor module can be configured to evaluate the cardiac information, to determine if the host's condition is improving or worsening. In another example, possible cardiac conditions can be separated/classified into levels or categories, which include certain criteria and are indicative of the severity of illness. For example, the NYHA and American College of Cardiology/American Heart Association staging systems for assessing heart failure severity can be implemented in the continuous cardiac marker sensor system. In some embodiments, the cardiac status can include at least one of improving cardiac health, declining cardiac health, stable cardiac health, ischemic heart disease, pericarditis, endocarditis, myocarditis, congestive cardiac failure, cardiogenic shock, acute coronary syndrome, alcoholic cardiomyopathy, coronary artery disease, congenital heart disease, ischemic cardiomyopathy, hypertensive cardiomyopathy, valvular cardiomyopathy, inflammatory cardiomyopathy, cardiomyopathy secondary to a systemic metabolic disease, dilated cardiomyopathy, hypertrophic cardiomyopathy, arrhythmogenic right ventricular cardiomyopathy, restrictive cardiomyopathy, noncompaction cardiomyopathy, congestive heart failure, valvular heart disease, and hypertensive heart disease, and the like.

In some embodiments, the processor module is configured to predict a cardiac event, such as but not limited to by evaluating (e.g., intelligently) the cardiac information, for example, by comparing a cardiac marker to a predetermined or programmed criterion/threshold. In one exemplary embodiment, the system is configured to continuously monitor the concentration of CK-MB, wherein if the CK-MB concentration is rapidly rising over a programmed period of time (e.g., 30-min., 1-hr, etc.), the system is configured to evaluate the cardiac information and predict if the host will experience a cardiac event (e.g., a myocardial infarction) within a second defined period of time, such as within the next 1-3 hours. In a further embodiment, the system is configured to alarm the medical staff (e.g., with an auditory alarm in the room, a text message delivered to the nurse's station, etc.), such that the medical staff can provide appropriate therapy to the host, thereby preventing or reducing the severity of a new heart attack. In some embodiments, the cardiac event includes but is not limited to a myocardial infarction, myocardial ischemia, myocardial rupture, pericarditis and cardiogenic shock.

In one exemplary embodiment, the system is configured with one or more user-selectable/user-definable formats for the cardiac output, such that the medical personnel can direct the system to output the cardiac information in one or more useful formats, such as by selection using a keyboard, a scroll menu or one or more dedicated buttons. In some exemplary embodiments, the system is configured with one or more locations for output, such that the medical personnel to select one or more locations where the cardiac information is to be output, such as but not limited to at the host's bedside and/or at a remote location, such as a nurse's station, the doctor's office, a clinical laboratory or medical records. Advantageously, configuring the system for cardiac information output at remote locations enables medical personnel to monitor and/or review the host's past, present and predicted cardiac status, including the host's current and historic cardiac information, without actually being in the room with the host. Similarly, in some embodiments, the system is configured with user selectable or user-definable information output (e.g., content), such that the medical personnel can select which cardiac information to output (e.g., concentration, change in concentration, and the like), for example.

In general, severely ill patients, such as cardiac patients in the ICU, are often connected to a plurality of monitoring/diagnostic/therapeutic devices concurrently. For convenience, any medical device in addition to a primary medical device such as the continuous cardiac marker sensor system of the preferred embodiments is referred to herein as a “secondary medical devices”. In some embodiments, the continuous cardiac marker sensor system of the preferred embodiments is configured to integrate with one or more secondary medical devices. For example, in some embodiments, the processor is configured to receive and process data from a secondary medical device (e.g., an ECG, a mechanical ventilator, a thermometer, an oxygen meter, a fluid delivery device, a pacing device (e.g., intra-aortic balloon pump), cardiac leads, a Doppler monitor, and the like), such as to provide secondary medical device information (e.g., for processing or display in the communication device). In some embodiments, the processor is configured to process the secondary medical device information when determining and/or predicting the cardiac status of a host, and/or predicting a cardiac event. In one exemplary embodiment, the continuous cardiac marker sensor system is configured to monitor a cardiac marker (e.g., CK-MB) concentration (e.g., of a host) and to receive data from an ECG (e.g., that is monitoring the host). In a further exemplary embodiment, the system is configured to evaluate data from the secondary medical device (e.g., the ECG indicated improved heart function), in addition to the continuous cardiac marker sensor system data (e.g., the CK-MB levels have reduced 4-fold), and predict a time period to a “mile-stone” level of recovery, at which the patient's therapy can be modified, such as by removing an intra-aortic balloon pump, weaning of a ventilator or modifying medicament delivery, for example. In some embodiments, the processor is configured to provide a therapy (e.g., recommendations and/or instructions) to medical personnel and/or to a secondary medical device, based on the current and/or predicted cardiac status/event. For example, in some circumstances, the system is configured to provide step-by-step instructions to the medical personnel, for performing a therapy, such as but not limited to increasing or decreasing the rate of medicament delivered. In another example, in some circumstances, the system is configured to provide instructions to an integrated secondary medical device, such as a medicament pump, to deliver the medicament as a faster or slower rate.

In preferred embodiments, the system is configured to output (e.g., display) information from a secondary medical device 120, such as on the system's user interface 216. For example, the system can be configured to receive data from an ECG (e.g., that is monitoring the same host as the continuous cardiac marker sensor system) and display the ECG output on the system's user interface 216. Similarly, the communication device can be configured to display information from other secondary medical devices, such as but not limited to an infusion pump, a ventilator, a temperature monitor, a cardiac pacing device, an oxygen monitor, and the like. Alternatively or additionally, the communication device can be configured to output the cardiac information to a secondary medical device, such that the output of continuous cardiac marker sensor system is provided to medical care personnel on the secondary medical device. In one exemplary embodiment, the system is configured to display the cardiac information on the user interface of a secondary medical device (e.g., a display or monitor). For example, in some circumstances, the system is configured to display cardiac information (e.g., current cardiac marker concentration, trends in cardiac marker concentration, level of host cardiac status, and the like) on the monitor/display screen of an ECG that is concurrently monitoring a patient.

In some embodiments, the communication device is configured to transmit instructions to a secondary medical device, such as in response to processing of sensor data. For example, in some circumstances, wherein when the concentration of a cardiac marker (e.g., of the host) is increasing (or decreasing) and passes a threshold, the communication device is configured to instruct an infusion pump to modify a medication delivery, such as by instructing the pump to deliver the medicament at a faster (or slower) rate. In an exemplary embodiment, the system is configured to monitor the relative concentrations of 2, 3, 4 or more cardiac markers (e.g., fluctuations up and down) and to provide an alarm when one or more criteria have been met (e.g., a first cardiac marker must reach a first level, a second cardiac marker must reach a second level, and/or the changes in the concentrations must fit a predetermined pattern within a predetermined level, over a predetermined time period).

In some embodiments, the system is configured receive and process information from a first secondary medical device, and then to provide instructions (e.g., for a therapy) to another (e.g., second) secondary medical device. In some circumstances, the system is configured to receive ECG information from an ECG concurrently monitoring the host (e.g., monitoring the same host (at the same time) as the continuous cardiac marker sensor system), and then to provide instructions to a medicament pump (e.g., concurrently providing medicament to the host) to modify the medicament delivery rate (e.g., increase or decrease the rate of delivery), for example. In some circumstances, the system is configured to receive information (e.g., data) from two, three, four or more secondary medical devices concurrently monitoring the host, to process the received information (e.g., in addition to the cardiac information from the continuous cardiac marker sensor system), and then to provide instructions to one or more of the secondary medical devices (e.g., to modify the function of the secondary medical device), to provide output on the system's user interface, and to provide one or more messages, instructions or alerts to medical personnel (e.g., either proximal to the sensor or remotely from the host).

In some embodiments, the sensor is configured to continuously measure a second analyte in vivo, and to provide a signal associated therewith. The second substance can include, but is not limited to, glucose, potassium, calcium, oxygen, carbon dioxide, liver enzymes, or any analyte listed in the “Definitions” herein. In some embodiments, the system is configured to receive and process data related to the concentration of the second analyte, in addition to processing the data from the cardiac marker sensor, to provide the host's cardiac status and/or to predict a cardiac status or a cardiac event. In a further embodiment, the system is configured to utilize the second analyte data, in conjunction with the cardiac information, to provide an output, such as relationship of the second analyte concentration to the cardiac marker concentration, changes in the concentrations, recommended therapies, messages to medical personnel, and the like. In one exemplary embodiment, the continuous cardiac marker sensor system is configured to receive and process data from a continuous glucose sensor (to provide glucose information), and then to output and/or display the cardiac and glucose information. In a further embodiment, the output can include instructions to medical personnel or to a secondary medical device. For example, in some circumstances, the secondary medical device is an insulin pump and the system is configured to process the glucose information and cardiac information, and then to provide therapy instructions to the insulin pump (e.g., low or increase a basal insulin dose rate, provide a bolus therapy, and the like) or to provide an alert to medical staff, such as but not limited to a bedside alarm and/or a remote alarm, such as at the nurse's station. While not wishing to be bound by theory, it is believed that tight control of certain analytes, such as but not limited to glucose, can be a critical factor in the successful recovery of the host. Any detection method known in the art, such as described elsewhere herein, can be used to detect the second analyte.

Cardiac Information

As described elsewhere herein, the continuous sensor is configured to continuously measure a concentration of a cardiac marker in vivo and to provide a signal associated therewith. The communication device processes the signal to obtain cardiac information and to output that cardiac information. The data/signal can be processed, such as by the processor, to provide output and/or display the cardiac information. In preferred embodiments, cardiac information can include but is not limited to the concentration (past, present or future) of a cardiac marker, changes in the concentration, acceleration of the change in concentration (e.g., whether the concentration is increasing, decreasing or substantially unchanging), peaks, and the “area under the curve” of a graph of cardiac marker concentration versus time. In some embodiments, cardiac information can include predicted marker concentration. In some embodiments, the system is configured to receive and process data and/or information from a second medical device, and to use/output these data/information in conjunction with the cardiac information.

Calibration of sensor data may or may not be required, depending upon a variety of factors, such as but not limited to the system's configuration, the analytes measured, the algorithms used, the desired output information, and the like. Accordingly, in some embodiments, the system is configured to calibrate the data received from the sensor, such as prior to processing to obtain cardiac information; while in other embodiment, the system is configured to process the data without calibration.

Since many cardiac markers do not generally appear in a person's blood until and unless cardiac injury has occurred, in some circumstances, accurate concentration (e.g., the true concentration) measurements are not generally necessary. Rather, the consistent measurement of the relative cardiac marker concentration and changes/fluctuations thereof (e.g., relative, uncalibrated output) provide sufficient and adequate cardiac information. For example, in some circumstances, the physician needs to know how the marker concentration fluctuates over time, such as relative to the marker concentrations when the host was first admitted (or at another time point). Accordingly, in some embodiments, the system is configured to consistently (e.g., capable of being reproduced) continuously measure the cardiac marker concentration, to process the sensor data and to output cardiac information without calibration, such that the output includes consistent continuous cardiac information (e.g., relative, uncalibrated output regarding fluctuations in the host's marker concentration).

In some circumstances, the actual cardiac marker concentration is necessary and/or preferred. Output of actual cardiac marker concentrations generally requires both accurate and consistent measurement of a cardiac marker concentration; and accurate measurement can require calibration. As used herein, the term “accurate” is a broad term, and is to be given its ordinary and customary meaning to a person of ordinary skill in the art (and it is not to be limited to a special or customized meaning), and refers without limitation to conforming exactly or almost exactly to fact or to a standard. Accordingly, in some embodiments, the system is configured to calibrate the sensor data using a defined relationship between sensor-generated measurements and a reference measurement that is meaningful to the user (e.g., analyte concentration in mg/dl). This defined relationship may be monitored to ensure that the continuous analyte sensor maintains a substantially accurate calibration and thereby continually provides meaningful values to a user. In some embodiments, the system is configured to calibrate the data (e.g., the sensor signal) using at least one reference data point. In some embodiments, the system is configured to calibrate the signal using at least one reference point for each of two or more cardiac markers. In some embodiments, the system is configured to define a relationship between a raw signal and a calibrated analyte value using calibration information (e.g., data) received from a single point or multipoint measurement device (e.g., configured to measure the concentration of a cardiac marker in a blood sample withdrawn from the host), such that the system provides calibrated output. One exemplary external cardiac marker measurement device that can be used to define the relationship between a raw signal and a calibrated analyte value (e.g., provide one or more reference data points) is the Triage® Meter (Biosite, Inc., San Diego, Calif., USA), which can measure BNP, CK-MB, Myoglobin, cTnI, and/or D-dimer.

In some circumstances, sensitivity and/or baseline of the calibration can be subject to changes that occur in vivo over time (for example, hours to months), requiring updates to the calibration. In some embodiments, certain physical properties that influence diffusion or transport of molecules to the electrode's electroactive surfaces (e.g., through a membrane) can alter the sensitivity (and/or baseline) of calibration. Physical properties that can alter the transport of molecules include, but are not limited to, blockage of the sensor's surface area due to IV-specific properties, protein build-up (e.g., biofouling), some medications delivered to the host, and the like.

Accordingly, in one aspect of the preferred embodiments, systems and methods are provided for measuring changes in sensitivity, also referred to as changes in solute transport or biointerface changes, of a continuous analyte sensor (e.g., a continuous cardiac marker analyte sensor) associated (e.g., exposed to the host's blood stream) with a host over a time period. Preferably, the sensitivity measurement is a signal obtained by measuring a constant analyte other than the analyte being measured by the continuous analyte sensor. For example, in a continuous cardiac marker sensor, a non-cardiac marker constant analyte is measured. In embodiments wherein the sensor includes a membrane system, the signal is measured beneath the membrane system on the continuous cardiac marker sensor. While not wishing to be bound by theory, it is believed that by monitoring the sensitivity over a time period, a change associated with solute transport (e.g., through a membrane system) can be measured and used as an indication of a sensitivity change in the analyte measurement. In other words, a biointerface monitor is provided, which is capable of monitoring changes in the biointerface surrounding an implantable device, thereby enabling the measurement of sensitivity changes of an analyte sensor over time.

In some embodiments, the continuous cardiac marker sensor 100 is provided with an auxiliary electrode (not shown) configured as a transport-measuring electrode disposed beneath the sensor's membrane system. The transport-measuring electrode can be configured to measure any of a number of substantially constant analytes or factors, such that a change measured by the transport-measuring electrode can be used to indicate a change in solute (for example, one or more cardiac markers) transport to the membrane system. Some examples of substantially constant analytes or factors that can be measured include, but are not limited to, oxygen, carboxylic acids (such as urea), amino acids, hydrogen, pH, chloride, baseline, or the like. Thus, the transport-measuring electrode provides an independent measure of changes in solute transport to the membrane, and thus sensitivity changes over time.

In some embodiments, the transport-measuring electrode measures analytes similar to the analyte being measured by the analyte sensor. For example, in some embodiments of a continuous cardiac marker sensor, water-soluble analytes are believed to better represent the changes in sensitivity to cardiac marker's over time than non-water soluble analytes (due to the water-solubility of cardiac markers), however relevant information may be ascertained from a variety of molecules. Although some specific examples are described herein, one skilled in the art appreciates a variety of implementations of sensitivity measurements that can be used as to qualify or quantify solute transport through the biointerface of the analyte sensor.

In one embodiment of a continuous cardiac marker sensor, the transport-measuring electrode is configured to measure urea, which is a water-soluble constant analyte. In one exemplary implementation wherein urea is directly measured by the transport-measuring electrode, the cardiac marker sensor comprises a membrane system, however, it does not include an active interference domain or active enzyme directly above the transport-measuring electrode, thereby allowing the urea to pass through the membrane system to the electroactive surface for measurement thereon. In one alternative exemplary implementation wherein urea is indirectly measured by the transport-measuring electrode, the cardiac marker sensor comprises a membrane system, and further includes an active uricase oxidase domain located directly above the transport-measuring electrode, thereby allowing the urea to react at the enzyme and produce hydrogen peroxide, which can be measured at the electroactive surface thereon.

In some embodiments, the system is configured to output the cardiac information in one or more formats, such as but not limited to in numeric and/or graphical representation, and/or as text. For example, in one embodiment, the system is configured to display the current cardiac marker concentration as a numeric value that can be easily understood by the medical personnel, such as but not limited to in mg/dl, μl/dl and the like. In another embodiment, the system is configured to display the cardiac marker concentrations measured over a given period of time as a graph. In still another embodiment, the system is configured to display an alarm as blinking red text that says “ALARM,” “ALERT” or “WARNING.” In a further embodiment, the system is configured to make beeping, buzzing and/or ringing sounds in conjunction with displaying the alarm text.

### Methods of Use

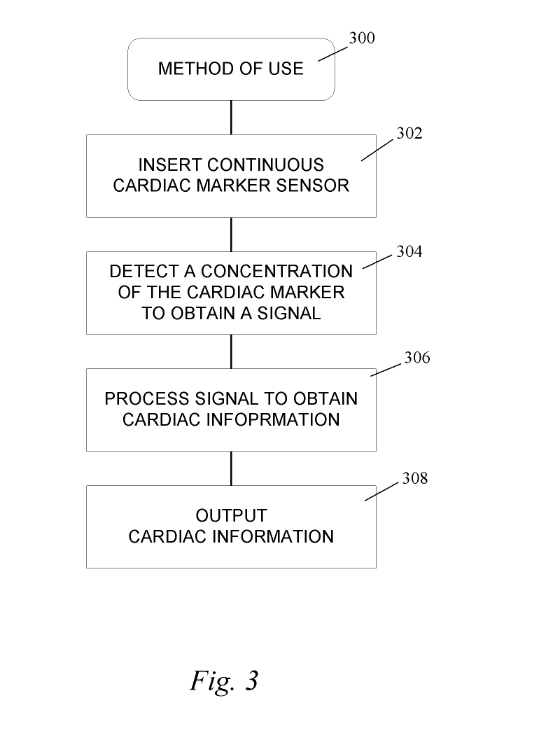

FIG. 3 is a block diagram illustrating a method of use 300 of the system 10 in one embodiment.

At block 302, a continuous cardiac marker sensor 100 is inserted into the circulatory system of the host, such as by using a vascular access device. Description of a continuous cardiac marker sensor can be found in the section entitled “Continuous Analyte Sensor.” A description of vascular access devices can be found in the section entitled “Vascular Access Device.” In some embodiments, the sensor system is configured with a second sensor configured to measure a second analyte, as discussed elsewhere herein. Additional descriptions of devices and methods of use can be found in U.S. Patent Publication No. US-2008-0119703-A1, U.S. Patent Publication No. US-2008-0119704-A1, U.S. Patent Publication No. US-2008-0119706-A1, U.S. Patent Publication No. US-2008-0108942-A1, U.S. Patent Publication No. US-2008-0086042-A1, U.S. Patent Publication No. US-2008-0086044-A1, and U.S. Patent Publication No. US-2008-0086273-A1, each of which is incorporated herein by reference in its entirety.

At block 304, the sensor measures a concentration of a cardiac marker to obtain a signal. As described elsewhere herein, the continuous cardiac marker sensor can be configured to measure at least one of troponin (cTnT, cTnI, TnC), creatine kinase MB (CK-MB), aspartate transaminase (AST), lactate dehydrogenase (LDH), myoglobin (MB), alanine transaminase (ALT), aspartate transaminase (AST), alkaline phosphatase (ALP), albumin (Alb), gamma glutamyl transpeptidase (GGT), high sensitive C-reactive protein (hsCRP), heart type fatty acid binding protein (H-FABP), myeloperoxidase (MPO), brain natriuretic peptide (BNP), P-selectin (soluble and membrane bound), soluble CD40 ligand (sCD40L), glycoprotein IIb/IIIa (GPIIb/IIIa), prothrombin fragment 1.2 (PTF1.2), D-dimer (DD), thrombin-antithrombin II (TAT), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), interleukin-1 receptor family/ST2, interleukin 6 (IL-6), interleukin 18 (IL-18), placental growth factor (PIGF), pregnancy-associated plasma protein A (PAPP-A), glutathione peroxidase, plasma thioredoxin, Cystatin C, serum deoxyribonuclease I, and ATP/ADP, total bilirubin (TBIL) and direct bilirubin. In some embodiments, cardiac markers include but are not limited to non-protein substances, such as serum potassium (K⁺) and calcium (Ca²⁺), some carbohydrates, lipids and nucleic acids, and the like. Additional markers of cardiac status/health include but are not limited to blood gases (O₂, CO₂), liver function tests, and glucose tests.

At block 306, the processor module processes the signal to obtain cardiac information. Cardiac information includes but is not limited to the concentration of the cardiac marker (e.g., mg/dl, μ/dl, etc.), changes in concentration (e.g., mg/dl/min) and the direction of change (e.g., increasing, decreasing), peaks and valleys (e.g., maximum and minimum concentrations over the past several minutes, hours or days, or since the host was admitted, since surgery was completed, and the like), as well as the “area under the curve” of a graph of marker concentration versus time. In some embodiments, changes in marker concentration are evaluated to determine the host's cardiac status (e.g., level 1, 2 or 3 on a triage scale), his cardiac health (e.g., morbidity and mortality risks), and predict a cardiac event (e.g., reinfarction or ischemia is worsening). Additionally, information such as “area under the curve” is indicative of the extent of cardiac damage already incurred (e.g., 5, 10, 20% or more of the heart was damaged by the previous myocardial infarction). In some embodiments, data from an operably connected secondary medical device (or second analyte sensor) can be received and is processed at block 306 to obtain cardiac information and/or information related thereto. Accordingly, processing can include providing a cardiac status or cardiac health. In some embodiments, processing includes predicting a future cardiac status. For example, information processed from the cardiac marker sensor data can be used to predict that the host's cardiac status will be improved. In some embodiments, predicted cardiac status can include a level. In some embodiments, processing includes predicting a time to a cardiac event (or a level thereof), such as described elsewhere herein.

At block 308, the cardiac information is output, such as by displaying the cardiac information. In some embodiments, the information can be displayed via the user interface 216. In some embodiments, information can be output on a communication device proximal to and/or remote from the sensor. For example, the cardiac information can be provided on a display on a communication device physically connected to a vascular access device, located on the host's bedside, at the nurse's station, or carried by the physician. In some embodiments, output of information can include providing therapy recommendations/instructions and/or one or more alarms. Additionally, in some embodiments, the system is configured to provide the output on an operably connect secondary medical device, such as on the user interface (e.g., a display) of an ECG, a fluid pump, a ventilator, a pressure monitor, a pacing device, and the like.

Methods and devices that are suitable for use in conjunction with aspects of the preferred embodiments are disclosed in U.S. Pat. No. 4,994,167; U.S. Pat. No. 4,757,022; U.S. Pat. No. 6,001,067; U.S. Pat. No. 6,741,877; U.S. Pat. No. 6,702,857; U.S. Pat. No. 6,558,321; U.S. Pat. No. 6,931,327; U.S. Pat. No. 6,862,465; U.S. Pat. No. 7,074,307; U.S. Pat. No. 7,081,195; U.S. Pat. No. 7,108,778; U.S. Pat. No. 7,110,803; U.S. Pat. No. 7,192,450; U.S. Pat. No. 7,226,978; U.S. Pat. No. 7,310,544; U.S. Pat. No. 7,364,592; U.S. Pat. No. 7,366,556; U.S. Pat. No. 7,424,318; U.S. Pat. No. 7,471,972; U.S. Pat. No. 7,460,898; and U.S. Pat. No. 7,467,003.

Methods and devices that are suitable for use in conjunction with aspects of the preferred embodiments are disclosed in U.S. Patent Publication No. US-2005-0143635-A1; U.S. Patent Publication No. US-2005-0181012-A1; U.S. Patent Publication No. US-2005-0177036-A1; U.S. Patent Publication No. US-2005-0124873-A1; U.S. Patent Publication No. US-2005-0115832-A1; U.S. Patent Publication No. US-2005-0245799-A1; U.S. Patent Publication No. US-2005-0245795-A1; U.S. Patent Publication No. US-2005-0242479-A1; U.S. Patent Publication No. US-2005-0182451-A1; U.S. Patent Publication No. US-2005-0056552-A1; U.S. Patent Publication No. US-2005-0192557-A1; U.S. Patent Publication No. US-2005-0154271-A1; U.S. Patent Publication No. US-2004-0199059-A1; U.S. Patent Publication No. US-2005-0054909-A1; U.S. Patent Publication No. US-2005-0051427-A1; U.S. Patent Publication No. US-2003-0032874-A1; U.S. Patent Publication No. US-2005-0203360-A1; U.S. Patent Publication No. US-2005-0090607-A1; U.S. Patent Publication No. US-2005-0187720-A1; U.S. Patent Publication No. US-2005-0161346-A1; U.S. Patent Publication No. US-2006-0015020-A1; U.S. Patent Publication No. US-2005-0043598-A1; U.S. Patent Publication No. US-2005-0033132-A1; U.S. Patent Publication No. US-2005-0031689-A1; U.S. Patent Publication No. US-2004-0186362-A1; U.S. Patent Publication No. US-2005-0027463-A1; U.S. Patent Publication No. US-2005-0027181-A1; U.S. Patent Publication No. US-2005-0027180-A1; U.S. Patent Publication No. US-2006-0020187-A1; U.S. Patent Publication No. US-2006-0036142-A1; U.S. Patent Publication No. US-2006-0020192-A1; U.S. Patent Publication No. US-2006-0036143-A1; U.S. Patent Publication No. US-2006-0036140-A1; U.S. Patent Publication No. US-2006-0019327-A1; U.S. Patent Publication No. US-2006-0020186-A1; U.S. Patent Publication No. US-2006-0036139-A1; U.S. Patent Publication No. US-2006-0020191-A1; U.S. Patent Publication No. US-2006-0020188-A1; U.S. Patent Publication No. US-2006-0036141-A1; U.S. Patent Publication No. US-2006-0020190-A1; U.S. Patent Publication No. US-2006-0036145-A1; U.S. Patent Publication No. US-2006-0036144-A1; U.S. Patent Publication No. US-2006-0016700-A1; U.S. Patent Publication No. US-2006-0142651-A1; U.S. Patent Publication No. US-2006-0086624-A1; U.S. Patent Publication No. US-2006-0068208-A1; U.S. Patent Publication No. US-2006-0040402-A1; U.S. Patent Publication No. US-2006-0036142-A1; U.S. Patent Publication No. US-2006-0036141-A1; U.S. Patent Publication No. US-2006-0036143-A1; U.S. Patent Publication No. US-2006-0036140-A1; U.S. Patent Publication No. US-2006-0036139-A1; U.S. Patent Publication No. US-2006-0142651-A1; U.S. Patent Publication No. US-2006-0036145-A1; U.S. Patent Publication No. US-2006-0036144-A1; U.S. Patent Publication No. US-2006-0200022-A1; U.S. Patent Publication No. US-2006-0198864-A1; U.S. Patent Publication No. US-2006-0200019-A1; U.S. Patent Publication No. US-2006-0189856-A1; U.S. Patent Publication No. US-2006-0200020-A1; U.S. Patent Publication No. US-2006-0200970-A1; U.S. Patent Publication No. US-2006-0183984-A1; U.S. Patent Publication No. US-2006-0183985-A1; U.S. Patent Publication No. US-2006-0195029-A1; U.S. Patent Publication No. US-2006-0229512-A1; U.S. Patent Publication No. US-2006-0222566-A1; U.S. Patent Publication No. US-2007-0032706-A1; U.S. Patent Publication No. US-2007-0016381-A1; U.S. Patent Publication No. US-2007-0027370-A1; U.S. Patent Publication No. US-2007-0032718-A1; U.S. Patent Publication No. US-2007-0059196-A1; U.S. Patent Publication No. US-2007-0066873-A1; U.S. Patent Publication No. US-2007-0197890-A1; U.S. Patent Publication No. US-2007-0173710-A1; U.S. Patent Publication No. US-2007-0163880-A1; U.S. Patent Publication No. US-2007-0203966-A1; U.S. Patent Publication No. US-2007-0213611-A1; U.S. Patent Publication No. US-2007-0232879-A1; U.S. Patent Publication No. US-2007-0235331-A1; U.S. Patent Publication No. US-2008-0021666-A1; U.S. Patent Publication No. US-2008-0033254-A1; U.S. Patent Publication No. US-2008-0045824-A1; U.S. Patent Publication No. US-2008-0071156-A1; U.S. Patent Publication No. US-2008-0086042-A1; U.S. Patent Publication No. US-2008-0086044-A1; U.S. Patent Publication No. US-2008-0086273-A1; U.S. Patent Publication No. US-2008-0083617-A1; U.S. Patent Publication No. US-2008-0119703-A1; U.S. Patent Publication No. US-2008-0119704-A1; U.S. Patent Publication No. US-2008-0119706-A1; U.S. Patent Publication No. US-2008-0194936-A1; U.S. Patent Publication No. US-2008-0194937-A1; U.S. Patent Publication No. US-2008-0195967-A1; U.S. Patent Publication No. US-2008-0183061-A1; U.S. Patent Publication No. US-2008-0183399-A1; U.S. Patent Publication No. US-2008-0189051-A1; U.S. Patent Publication No. US-2008-0214918-A1; U.S. Patent Publication No. US-2008-0194938-A1; U.S. Patent Publication No. US-2008-0214915-A1; U.S. Patent Publication No. US-2008-0194935-A1; U.S. Patent Publication No. US-2008-0188731-A1; U.S. Patent Publication No. US-2008-0242961-A1; U.S. Patent Publication No. US-2008-0208025-A1; U.S. Patent Publication No. US-2008-0197024-A1; U.S. Patent Publication No. US-2008-0200788-A1; U.S. Patent Publication No. US-2008-0200789-A1; U.S. Patent Publication No. US-2008-0200791-A1; U.S. Patent Publication No. US-2008-0228054-A1; U.S. Patent Publication No. US-2008-0228051-A1; U.S. Patent Publication No. US-2008-0262469-A1; U.S. Patent Publication No. US-2008-0108942-A1; U.S. Patent Publication No. US-2008-0306368-A1; U.S. Patent Publication No. US-2009-0012379-A1; U.S. Patent Publication No. US-2008-0287765-A1; U.S. Patent Publication No. US-2008-0287764-A1; U.S. Patent Publication No. US-2008-0287766-A1; U.S. Patent Publication No. US-2008-0275313-A1; U.S. Patent Publication No. US-2008-0296155-A1; U.S. Patent Publication No. US-2008-0306434-A1; U.S. Patent Publication No. US-2008-0306444-A1; U.S. Patent Publication No. US-2008-0306435-A1; and U.S. Patent Publication No. US-2009-0018424-A1.

Methods and devices that are suitable for use in conjunction with aspects of the preferred embodiments are disclosed in U.S. patent application Ser. No. 09/447,227 filed Nov. 22, 1999 and entitled “DEVICE AND METHOD FOR DETERMINING ANALYTE LEVELS”; U.S. patent application Ser. No. 11/654,135 filed Jan. 17, 2007 and entitled “POROUS MEMBRANES FOR USE WITH IMPLANTABLE DEVICES”; U.S. patent application Ser. No. 11/654,140 filed Jan. 17, 2007 and entitled “MEMBRANES FOR AN ANALYTE SENSOR”; U.S. patent application Ser. No. 12/103,594 filed Apr. 15, 2008 and entitled “BIOINTERFACE WITH MACRO- AND MICRO-ARCHITECTURE”; U.S. patent application Ser. No. 12/055,098 filed Mar. 25, 2008 and entitled “ANALYTE SENSOR”; U.S. patent application Ser. No. 12/054,953 filed Mar. 25, 2008 and entitled “ANALYTE SENSOR”; U.S. patent application Ser. No. 12/133,789 filed Jun. 5, 2008 and entitled “INTEGRATED MEDICAMENT DELIVERY DEVICE FOR USE WITH CONTINUOUS ANALYTE SENSOR”; U.S. patent application Ser. No. 12/139,305 filed Jun. 13, 2008 and entitled “ELECTRODE SYSTEMS FOR ELECTROCHEMICAL SENSORS”; U.S. patent application Ser. No. 12/182,073 filed Jul. 29, 2008 and entitled “INTEGRATED RECEIVER FOR CONTINUOUS ANALYTE SENSOR”; U.S. patent application Ser. No. 12/247,137 filed Oct. 7, 2008 and entitled “IMPLANTABLE ANALYTE SENSOR”; U.S. patent application Ser. No. 12/250,918 filed Oct. 14, 2008 and entitled “ANALYTE SENSOR”; U.S. patent application Ser. No. 12/253,125 filed Oct. 16, 2008 and entitled “SIGNAL PROCESSING FOR CONTINUOUS ANALYTE SENSOR”; U.S. patent application Ser. No. 12/253,120 filed Oct. 16, 2008 and entitled “SIGNAL PROCESSING FOR CONTINUOUS ANALYTE SENSOR”; U.S. patent application Ser. No. 12/253,064 filed Oct. 16, 2008 and entitled “SIGNAL PROCESSING FOR CONTINUOUS ANALYTE SENSOR”; U.S. patent application Ser. No. 12/252,996 filed Oct. 16, 2008 and entitled “SIGNAL PROCESSING FOR CONTINUOUS ANALYTE SENSOR”; U.S. patent application Ser. No. 12/252,967 filed Oct. 16, 2008 and entitled “SIGNAL PROCESSING FOR CONTINUOUS ANALYTE SENSOR”; U.S. patent application Ser. No. 12/252,952 filed Oct. 16, 2008 and entitled “SIGNAL PROCESSING FOR CONTINUOUS ANALYTE SENSOR”; U.S. patent application Ser. No. 12/260,017 filed Oct. 28, 2008 and entitled “SENSOR HEAD FOR USE WITH IMPLANTABLE DEVICES”; U.S. patent application Ser. No. 12/258,320 filed Oct. 24, 2008 and entitled “SYSTEMS AND METHODS FOR PROCESSING SENSOR DATA”; U.S. patent application Ser. No. 12/263,993 filed Nov. 3, 2008 and entitled “SIGNAL PROCESSING FOR CONTINUOUS ANALYTE SENSOR”; U.S. patent application Ser. No. 12/264,835 filed Nov. 4, 2008 and entitled “IMPLANTABLE ANALYTE SENSOR”; U.S. patent application Ser. No. 12/258,235 filed Oct. 24, 2008 and entitled “SYSTEMS AND METHODS FOR PROCESSING SENSOR DATA”; U.S. patent application Ser. No. 12/258,345 filed Oct. 24, 2008 and entitled “SYSTEMS AND METHODS FOR PROCESSING SENSOR DATA”; U.S. patent application Ser. No. 12/258,325 filed Oct. 24, 2008 and entitled “SYSTEMS AND METHODS FOR PROCESSING SENSOR DATA”; U.S. patent application Ser. No. 12/258,318 filed Oct. 24, 2008 and entitled “SYSTEMS AND METHODS FOR PROCESSING SENSOR DATA”; U.S. patent application Ser. No. 12/258,335 filed Oct. 24, 2008 and entitled “SYSTEMS AND METHODS FOR PROCESSING SENSOR DATA”; U.S. patent application Ser. No. 12/264,160 filed Nov. 3, 2008 and entitled “DUAL ELECTRODE SYSTEM FOR A CONTINUOUS ANALYTE SENSOR”; U.S. patent application Ser. No. 12/267,542 filed Nov. 7, 2008 and entitled “ANALYTE SENSOR”; U.S. patent application Ser. No. 12/353,787 filed Jan. 14, 2009 and entitled “SYSTEMS AND METHODS FOR REPLACING SIGNAL ARTIFACTS IN A GLUCOSE SENSOR DATA STREAM”; U.S. patent application Ser. No. 12/353,799 filed Jan. 14, 2009 and entitled “SYSTEMS AND METHODS FOR REPLACING SIGNAL ARTIFACTS IN A GLUCOSE SENSOR DATA STREAM”; U.S. patent application Ser. No. 12/263,993 filed Nov. 3, 2008 and entitled “SIGNAL PROCESSING FOR CONTINUOUS ANALYTE SENSOR”; U.S. patent application Ser. No. 12/335,403 filed Dec. 15, 2008 and entitled “DUAL ELECTRODE SYSTEM FOR A CONTINUOUS ANALYTE SENSOR”; U.S. patent application Ser. No. 12/267,518 filed Nov. 7, 2008 and entitled “ANALYTE SENSOR”; U.S. patent application Ser. No. 12/264,835 filed Nov. 4, 2008 and entitled “IMPLANTABLE ANALYTE SENSOR”; U.S. patent application Ser. No. 12/273,359 filed Nov. 18, 2008 and entitled “TRANSCUTANEOUS ANALYTE SENSOR”; U.S. patent application Ser. No. 12/329,496 filed Dec. 5, 2008 and entitled “TRANSCUTANEOUS ANALYTE SENSOR”; U.S. patent application Ser. No. 12/359,207 filed Jan. 23, 2008 and entitled “TRANSCUTANEOUS ANALYTE SENSOR”; U.S. patent application Ser. No. 12/353,870 filed Jan. 14, 2009 and entitled “TRANSCUTANEOUS ANALYTE SENSOR”; U.S. patent application Ser. No. 12/267,525 filed Nov. 7, 2008 and entitled “ANALYTE SENSOR”; U.S. patent application Ser. No. 12/267,548 filed Nov. 7, 2008 and entitled “ANALYTE SENSOR”; U.S. patent application Ser. No. 12/267,547 filed Nov. 7, 2008 and entitled “ANALYTE SENSOR”; U.S. patent application Ser. No. 12/267,546 filed Nov. 7, 2008 and entitled “ANALYTE SENSOR”; U.S. patent application Ser. No. 12/267,544 filed Nov. 7, 2008 and entitled “ANALYTE SENSOR”; U.S. patent application Ser. No. 12/267,545 filed Nov. 7, 2008 and entitled “ANALYTE SENSOR”; U.S. patent application Ser. No. 12/267,494 filed Nov. 7, 2008 and entitled “INTEGRATED DEVICE FOR CONTINUOUS IN VIVO ANALYTE DETECTION AND SIMULTANEOUS CONTROL OF AN INFUSION DEVICE”; and U.S. patent application Ser. No. 12/267,531 filed Nov. 7, 2008 and entitled “ANALYTE SENSOR.”

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. A system for continuously detecting a cardiac marker, comprising:
a continuous sensor configured to continuously, continually, and/or intermittently measure a concentration of a cardiac marker in vivo and provide a signal associated therewith; and
a communication device comprising a processor module configured to process the signal to obtain cardiac information, wherein the communication device is configured to output the cardiac information.

2. The system of claim 1, wherein the cardiac marker is selected from the group consisting of creatine kinase MB, cardiac troponin T, cardiac troponin I, troponin C, aspartate transaminase, lactate dehydrogenase, myoglobin, alanine transaminase, alkaline phosphatase, albumin, ischemia-modified albumin, myeloperoxidase, glycogen phosphorylase isoenzyme BB, brain natriuretic peptide, N-terminal pro-natriuretic peptide, monocyte chemo attractive protein, gamma glutamyl transpeptidase, high sensitive C-reactive protein, heart type fatty acid binding protein, P-selectin, soluble CD40 ligand, glycoprotein IIb/IIIa, prothrombin fragment 1.2, D-dimer, thrombin-antithrombin II, beta-thromboglobulin, platelet factor 4, platelet/endothelial cell adhesion molecule 1, soluble fibrin, glycogen phosphorylase-BB, thrombus precursor protein, interleukin-1 receptor family/ST2, interleukin 6, interleukin 18, placental growth factor, pregnancy-associated plasma protein A, glutathione peroxidase, plasma thioredoxin, Cystatin C, serum deoxyribonuclease I, ATP/ADP, total bilirubin, direct bilirubin, potassium, calcium, and combinations thereof.

3. The system of claim 1, wherein the cardiac information is selected from the group consisting of a cardiac marker concentration, a change in cardiac marker concentration, an acceleration of cardiac marker concentration change, an area under the curve of a plot of time versus cardiac marker concentration, and combinations thereof.

4. The system of claim 1, wherein the communication device is configured to provide one or more alarms indicative of cardiac health.

5. The system of claim 4, wherein the processor module is configured to trigger the alarm when the cardiac marker concentration meets a criterion.

6. The system of claim 1, wherein the processor module is configured to provide a cardiac status.

7. The system of claim 6, wherein the cardiac status comprises a level of cardiac status.

8. The system of claim 6, wherein the processor module is configured to predict a cardiac status.

9. The system of claim 6, wherein the cardiac status is selected from the group consisting of improving cardiac health, declining cardiac health, stable cardiac health, ischemic heart disease, pericarditis, endocarditis, myocarditis, congestive cardiac failure, cardiogenic shock, acute coronary syndrome, alcoholic cardiomyopathy, coronary artery disease, congenital heart disease, ischemic cardiomyopathy, hypertensive cardiomyopathy, valvular cardiomyopathy, inflammatory cardiomyopathy, cardiomyopathy secondary to a systemic metabolic disease, dilated cardiomyopathy, hypertrophic cardiomyopathy, arrhythmogenic right ventricular cardiomyopathy, restrictive cardiomyopathy, noncompaction cardiomyopathy, congestive heart failure, valvular heart disease, hypertensive heart disease, and combinations thereof.

10. The system of claim 6, wherein the processor module is configured to predict a cardiac event.

11. The system of claim 10, wherein the cardiac event is selected from the group consisting of myocardial infarction, myocardial ischemic attack, unstable angina, acute coronary syndrome, myocardial rupture, endocarditis, pericarditis, cardiogenic shock, and combinations thereof.

12. The system of claim 1, further comprising a vascular access device configured for insertion into at least one of a circulatory system of the host and an extracorporeal blood circulation device.

13. The system of claim 1, wherein the sensor is further configured to continuously, continually, and/or intermittently measure a second substance in vivo and to provide a signal associated therewith.

14. The system of claim 13, wherein the second substance is selected from the group consisting of glucose, potassium, calcium, oxygen, carbon dioxide, and liver enzymes.

15. The system of claim 1, wherein the communication device is configured to receive and process data from a secondary medical device.

16. The system of claim 15, wherein the secondary medical device is selected from the group consisting of an electrocardiograph, an oxygen monitor, a fluid delivery device, a pacing device, leads, a mechanical ventilator, an extracorporeal membrane oxygenator, a cardiac output monitor, a blood pressure monitor, a central venous pressure monitor, a pulmonary capillary wedge pressure monitor, an intra-aortic balloon pump, an end-tidal carbon dioxide monitor, an intra-cranial pressure monitor, a Doppler monitor, a thermometer, a hemodynamic monitor, a patient monitor, and combinations thereof.

17. The system of claim 15, wherein the communication device is configured to display data from the secondary medical device.

18. The system of claim 1, wherein the communication device is configured to transmit instructions to a secondary medical device.

19. The system of claim 18, wherein the secondary medical device displays the cardiac information.

20. The system of claim 1, wherein the communication device comprises a user interface configured to display the cardiac information.

21. The system of claim 20, wherein the user interface is remote.

22. The system of claim 20, wherein the user interface is configured to provide an alarm.

23. The system of claim 1, wherein the communication device comprises a component of a secondary medical device.

24. The system of claim 23, wherein the secondary medical device is selected from the group consisting of an electrocardiograph, an oxygen monitor, a fluid delivery device, a pacing device, leads, a mechanical ventilator, an extracorporeal membrane oxygenator, a cardiac output monitor, a blood pressure monitor, a central venous pressure monitor, a pulmonary capillary wedge pressure monitor, an intra-aortic balloon pump, an end-tidal carbon dioxide monitor, an intra-cranial pressure monitor, a Doppler monitor, a thermometer, a hemodynamic monitor, a patient monitor, and combinations thereof.

25. The system of claim 1, wherein the system is configured to calibrate the signal using at least one reference data point.

26. The system of claim 25, wherein the system is configured to calibrate the signal using at least one reference point for each of two or more cardiac markers.

27. A method for determining cardiac health of a host, comprising:
using a sensor to continuously, continually, and/or intermittently detect a concentration of a cardiac marker in vivo;
providing a signal associated with the concentration of the cardiac marker;
processing the signal to obtain cardiac information; and
outputting the cardiac information.

28. The method of claim 27, wherein the cardiac marker is selected from the group consisting of creatine kinase MB, cardiac troponin T, cardiac troponin I, troponin C, aspartate transaminase, lactate dehydrogenase, myoglobin, alanine transaminase, alkaline phosphatase, albumin, ischemia-modified albumin, myeloperoxidase, glycogen phosphorylase isoenzyme BB, brain natriuretic peptide, N-terminal pro-natriuretic peptide, monocyte chemo attractive protein, gamma glutamyl transpeptidase, high sensitive C-reactive protein, heart type fatty acid binding protein, P-selectin, soluble CD40 ligand, glycoprotein IIb/IIIa, prothrombin fragment 1.2, D-dimer, thrombin-antithrombin II, beta-thromboglobulin, platelet factor 4, platelet/endothelial cell adhesion molecule 1, soluble fibrin, glycogen phosphorylase-BB, thrombus precursor protein, interleukin-1 receptor family/ST2, interleukin 6, interleukin 18, placental growth factor, pregnancy-associated plasma protein A, glutathione peroxidase, plasma thioredoxin, Cystatin C, serum deoxyribonuclease I, ATP/ADP, total bilirubin, direct bilirubin, potassium, calcium, and combinations thereof.

29. The method of claim 27, wherein the processing step comprises providing a cardiac status.

30. The method of claim 29, wherein the cardiac status comprises a level of cardiac status.

31. The method of claim 29, wherein the processing step comprises predicting a future cardiac status.

32. The method of claim 27, wherein the processing step comprises predicting a cardiac event.

33. The method of claim 27, wherein the outputting step comprises displaying the cardiac information.

34. The method of claim 27, wherein the outputting step comprises providing one or more alarms.

35. The method of claim 27, further comprising the step of calibrating the signal.
